SEQUENCE LISTING (2 sequences)

SEQ ID NO: 1 - protein, 9 aa, Artificial Sequence
  Description of Artificial Sequence/note = Synthetic Construct
ASNENMETM

SEQ ID NO: 2 - protein, 10 aa, Artificial Sequence
  Description of Artificial Sequence/note = Synthetic Construct
SSLENFRAYV

(19) United States
(12) Patent Application Publication
(10) Pub. No.: US 2008/0038283 A1
(43) Pub. Date: Feb. 14, 2008

(54) Methods of Evaluating Efficacy of an Immune Response by Assesing Alpha-1 Integrin Expression

(76) Inventors: David J. Topham, Pittsford, NY (US); Steven J. Ray, Cary, NC (US)

Correspondence Address: NEEDLE & ROSENBERG, P.C., SUITE 1000, 999 PEACHTREE STREET, ATLANTA, GA, 30309-3915, US

(21) Appl. No.: 10/589,152

(22) PCT Filed: Feb. 16, 2005
(86) PCT No.: PCT/US05/04900

§ 371 Date: May 31, 2007

Related U.S. Application Data
(60) Provisional application No. 60/545,559, filed on Feb. 17, 2004.

(51) Int. Cl. A61K 35/12; A61K 39/00; A61P 29/00; A61P 31/04; A61P 31/06; A61P 31/10; A61P 31/12; A61P 31/16; A61P 31/18; A61P 31/22; A61P 33/00; A61P 33/02; A61P 35/00; A61P 35/02; C12N 5/06; C12Q 1/06; C12Q 1/70; G01N 33/53

(52) U.S. Cl. 424/184.1; 424/93.7; 435/34; 435/378; 435/5; 435/7.1

(57) ABSTRACT

Disclosed are compositions and methods evaluating efficacy of an immune response by assessing alpha-1 integrin, screening for, and manufacturing those therapeutic agents. Described herein the presence of VLA-1+ (positive) T cells indicates an effective immune response. Also disclosed are methods of screening for antigens that induce the production of VLA-1+ (positive) T cells, methods of isolating the antigens, and methods using the antigens identified in the screening methods to treat a subject with a disease.

This application claims benefit of U.S. Provisional Application No. 60/545,559 filed on Feb. 17, 2004, which is incorporated herein in its entirety.

This invention was made with government support under Grants NIH/NIA #RO1 AG021970-01, #P30 AG18254, NIH/NRSA training grant #1T32HL66988-02, and NIEHS grant ESO 1247 awarded by the National Institutes of Health. The government has certain rights in the invention.

### SUMMARY

A common feature of many infections is that many pathogen-specific memory T cells become established in diverse non-lymphoid tissues. The studies described herein show that the collagen-binding α1β1 integrin, VLA-1, is expressed by the majority of influenza-specific CD8 T cells recovered from non-lymphoid tissues during both the acute and memory phases of the immune response. This indicates that VLA-1 is responsible for retaining protective memory CD8 T cells in the lung and other tissues via attachment to the extracellular matrix. Described herein are methods of assessing the strength of an immune response to an antigen. Also described herein are methods of treating a subject using antigens identified through using the methods disclosed herein.

### I. BACKGROUND OF THE INVENTION

Effector/memory T cells in peripheral non-lymphoid tissues are important during secondary encounters with pathogens. Following primary exposure to infection, both CD4 and CD8 pathogen-specific T cells distribute to virtually all lymphoid and extralymphoid sites (Hogan, R. J., et al. (2001) J Immunol 166, 1813-1822; Marshall, D. R., et al. (2001) Proc Natl Acad Sci USA 98, 6313-6318; Masopust, D., et al. (2001) Science 291, 2413-2417; Reinhardt, R. L., et al. (2001) Nature 410, 101-105). For some tissues, such as skin and gut, the tissue-specific homing receptors have been defined (Butcher, E. C., and Picker, L. J. (1996) Science 272, 60-66; Kunkel, E. J., and Butcher, E. C. (2002) Immunity 16, 1-4). For other tissues, including the lung, no organ specific receptors have been described. In comparison to memory T cells isolated from central lymphoid sites, the T cells isolated from a variety of non-lymphoid tissues exhibit altered functional capacities, such as immediate cytotoxicity and cytokine secretion, suggesting that they are a distinct subset of effector/memory T cells (Masopust, D., et al. (2001) Science 291, 2413-2417; Reinhardt, R. L., et al. (2001) Nature 410, 101-105). However, a molecular mechanism that allows these cells to localize to non-lymphoid sites has not been described.

Respiratory infection with influenza virus is a major cause of illness and death in humans (Layer, W. G., et al. (2000) Perspect Biol Med 43, 173-192; Munoz, F. M., et al. (2000) Antiviral Res 46, 91-124; Webster, R. G. (1998) Emerg Infect Dis 4, 436-441). A variety of influenza serotypes exist in nature and are defined by the hemagglutinin (H) and neuraminidase (N) neutralization activity. Fortunately, most individuals have heterosubtype-specific cytotoxic and helper T cells that recognize conserved internal epitopes common to different serotypes (Liang, S., et al. (1994) J Immunol 152, 1653-1661). Thus while the individual may become re-infected with a novel influenza serotype, the severity of disease, duration of infection, and even mortality is reduced by an effective heterosubtype specific cellular immune response (Liang, S., et al. (1994) J Immunol 152, 1653-1661). Thus needed in the art is a means of promoting the retention of protective effector/memory CD8 T cells in the lung and other non-lymphoid organs.

### II. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows the expression of VLA-1 and A2B1 on CD4+ and CD8⁺ cells before and after infection with influenza A/HK/x31. Spleen (SPL), MLN, and BAL cells were harvested from B6 female mice prior to infection (A) or 8 days after intranasal infection with influenza A/HK/x31 (B-D). FIGS. 1A and 1B show that VLA-1 expression was analyzed on CD4 and CD8 cells subsequent to staining with anti-CD4-APC, anti-CD8-TC, and anti-VLA1-Alexa488 and gating on either CD4+ or CD8+ live lymphocytes. FIG. 1C shows VLA-1 expression on flu NP and PA specific CD8 T cells which were determined after staining BAL cells with anti-VLA1-Alexa488, D^{b}NP-PE or D^{b}PA-PE tetramers, and anti-CD8-TC and gating on the CD8+ cells. FIG. 1D shows the secretion of IFN-γ in response to either NP or PA peptides (10 μM) determined by intracellular cytokine staining with anti-VLA1-Alexa488, anti-IFN-γ PE, and anti-CD8-TC then gating on CD8+ cells. Numbers in the quadrants indicate the percent positive cells in that quadrant or region, with the number in parentheses indicating the percent VLA-1+ cells within the tetramer+ or IFN-γ+ subsets.

FIG. 2 shows the persistence of flu-specific VLA-1+ CD8 T cells in the lung. FIG. 2A shows that 14 days after infection of B6 mice with influenza A/X31, the BAL, MLN and spleen (SPL) were harvested and CD8+ T cells analyzed with D^{b}/NP-PE tetramer and anti-α1-Alexa488 Ha31/8 mAb. Values in the quadrants indicate the percent positive cells. The percentage of VLA-1+ cells among the tetramer+ cells is indicated by the numbers in parentheses. FIG. 2B shows the relative prevalence of VLA-1+ CD8+ D^{b}NP+ cells over the course of the infection as determined by flow cytometric analysis of BAL, MLN, and SPL cells subsequent to staining with anti-α1-Alexa488, D^{b}NP-PE tetramer, and anti-CD8-TC. FIG. 2C shows the BAL of day 8 X31 infected mice as analyzed for apoptosis by TUNEL staining of CD8+ VLA-1+ and CD8+ VLA-1− cells. Data are representative of four experiments. FIGS. 2D and 2E show, 53 days after infection with X31, CD8+ BAL cells analyzed by flow cytometry for VLA-1 and VLA-2 expression (D) and VLA-1 versus D^{v}NP or D^{b}PA tetramers among the CD8+ cells (E).

FIG. 3 shows the localization of T cells in the lungs of recovered mice to areas of collagen adjacent to conducting airways and activation markers on the VLA-1+ CD8 T cells. Thirty days after X31 infection, lungs were obtained from recovered animals for immunohistochemical analysis of collagen deposition and localization of CD8+ cells. Lungs were inflated with a 1:1 PBS/OCT mixture. FIGS. 3A-3D show histology and immunohistochemical staining. Serial sections of zinc-fixed lungs were prepared and stained with (A) trichrome to mark collagen deposits; (B) hematoxylin and eosin; (C) rat anti-CD8β, developed with biotinylated rabbit anti-rat secondary, Streptavidin-horse radish peroxidase and DAB (black) to identify T cells (hematoxylin counterstain); (D) dual CD8β immunohistochemical and trichrome collagen staining to demonstrate colocalization of CD8+ T cells and collagen in the peribronchial and perivascular connective tissue; (E) peripheral lymphocytes, isolated and stained for CD8 and VLA-1, after gating on the CD8+ VLA-1+ lymphocytes, the expression of CD44, CD62L, 1B11 (CD43) and CD69 analyzed after additional staining with activation marker specific mAb as described below. AV=alveolus, AW=airway, BV=blood vessel.

FIG. 4 shows that VLA-1 is not essential for recruitment of CD8 T cells to the infected lung. FIG. 4A shows B6 mice treated with Ha 31/8 mAb to block VLA-1 (open bars), or a control (Ha 4/8, solid bars). Treatment consisted of administering 250 μg of each antibody to each mouse beginning 3 days prior to infection and continuing on alternate days throughout the course of the experiment. Individual spleen and pooled BAL cells were harvested and counted from 5 mice at 7 and 10 days after infection and analyzed for the presence of CD8+ D^{b}NP tetramer+ cells by flow cytometry. Data are representative of 3 experiments, and the error bars indicate 1 standard deviation from the mean of the 5 mice within one experiment. *P>0.01. FIG. 4B shows day 6 X31-infected Thy1.1+ congenic recipients that received via i.v. transfer, 7×10⁵ BAL lymphocytes (75% VLA-1+) from day 8×31 infected Thy1.2+B6 mice treated with anti-α1 (Ha31/8) or control (Ha 4/8) mAb. BAL of the recipient mice was sampled 24 hours later for the presence of the donor Thy1.2+ T cells. Values in the quadrants indicate the percent positive cells in that quadrant. Numbers in parentheses indicate the percent of CD8+ cells that were VLA-1+ at the time of transfer. FIG. 4C shows influenza A/X31 immune B6 mice (3 months after X31 infection) treated with either Ha31/8 anti-VLA-1 or control Ha 4/8 mAb (250 μg ip) on days −5, −3, and −1 prior to sampling. Animals were then analyzed by flow cytometry for the presence of flu-specific CD8 T cells in the liver (average of 4 individual mice±std dev) and BAL (pooled from 4 mice) after CD8 and D^{b}/NP or D^{b}/PA tetramer staining.

FIG. 5 shows that VLA-1 blockade reduced the number of flu-specific CD8 T cells in the periphery and compromised secondary heterosubtypic immune protection. Influenza A/HK/x31 (H₃N₂) immune B6 mice (3 months after primary X31 infection) were treated with either Ha31/8 anti-α1 or control Ha 4/8 mAb (250 μg ip) on days −5, −3, and −1 prior to intranasal challenge with 3×10⁵ EID₅₀ lethal influenza A/PR8 (H1N1). FIG. 5A shows data from animals analyzed 6 days after PR8 challenge by flow cytometry for the presence of DbNP-specific CD8 T cells in the lung by BAL (pooled) after CD8 and D^{b}/NP tetramer staining. FIG. 5B shows data from animals monitored for survival after PR8 challenge (2 experiments×5 mice per group=10 total). Non-immune animals (n=5) were also monitored for morbidity after PR8 challenge. The mean survival time between the control and anti-VLA-1 treated groups was significantly different (p=0.01, unpaired T-test).

FIG. 6 shows that Alpha-1 integrin deficiency alters the distribution of NP-specific CD8 T cells and enhances susceptibility to secondary virus infection. Wild-type and alpha-1 integrin deficient B6 mice were primed with influenza A/HK/x31 (H₃N₂) as described. FIG. 6A shows data form animals three months after priming. The animals were sampled for the presence of flu-specific D^{b}/NP tetramer+ CD8 T cells in the BAL, lung tissue, MLN, and spleen. Three animals per group were analyzed, the BAL and lung tissue lymphocytes remaining after lavage were pooled, while the MLN and spleen samples were analyzed independently. Error bars indicate the standard deviation of the mean for the three animals. FIG. 6B shows that the mice remaining were challenged with influenza A/PR8 (H1N1) at 3×10⁴ or 3×10⁵ EID₅₀ per mouse in two independent experiments (5 per group in each experiment), and monitored for survival. Mortality in each group was similar in both experiments, so the data were combined into a single FIGURE. Comparison of the mean survival time between the two groups gave a p=0.10 by the unpaired T-test.

### III. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### DEFINITIONS

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

“Optional” or “optionally” means that the subsequently described event or circumstance may or may not occur, and that the description includes instances where said event or circumstance occurs and instances where it does not.

Methods of Screening

Disclosed herein are methods of assessing the sufficiency of an immune response in a subject comprising selecting a subject for determining the efficacy or sufficiency of the immune response to a selected antigen, introducing into the subject the antigen, collecting a tissue sample (for example, a peripheral non-lymphoid tissue including but not limited to peripheral b11od) from the subject, and detecting the presence of VLA-1+ (positive), antigen-specific T-cells in the sample, the presence of VLA-1+ (positive) antigen-specific T-cells indicating a sufficient immune response in the subject. Antigen-specificity can be assessed by the contacting of a T cell with a labeled MHC molecule presenting an antigenic peptide, wherein the MHC-antigen molecule can be in the form of a dimer or tetramer. The T cell-MHC-peptide combination can then be visualized by any method known in the art (e.g., flow cytometry or Immunohistochemistry). Antigen specificity can also be determined by stimulating T cells with the antigen an identifying those T cells that secrete a cytokine (e.g., IFN-γ, IL-2, IL-4, IL-10, or TNF-α) in response to the antigenic stimulation.

Very Late Antigen-1 (VLA-1) is a heterodimeric integrin comprised of an α1 and β1 chain. Thus VLA-1 is an α1 integrin. It is understood that throughout this application, when reference is made to VLA-1, reference is also being made to α1 integrin. Thus it is understood that any method disclosed herein that would apply to VLA-1 would also apply to only the α1 chain. For example, a positive staining for only the α1 integrin is considered herein to also be a positive stain for VLA-1. An example of a reagent that can be used to identify VLA-1 positive cells that actually identifies the α1 chain of VLA-1 is anti-CD49a antibody. Therefore, one specific embodiment of the disclosed methods is a method of assessing the efficacy or sufficiency of an immune response in a subject comprising introducing into the subject the antigen, collecting a tissue sample from the subject, and detecting the presence of VLA-1+ (positive), antigen-specific T-cells in the sample, wherein VLA-1 positive cells are identified by being positive for CD49a, and wherein the presence of VLA-1+ (positive) antigen-specific T-cells indicates a sufficient immune response in the subject.

“Efficacy,” “efficacious,” or “sufficiency” mean the ability to function as intended. For example, an “efficacious” immune response is a response that is able to afford the subject an acceptable degree of immune protection from the immunizing antigen. Thus, the present methods disclose methods of assessing the ability of an immune response to provide immune protection against future antigenic encounter. Traditionally, such methods involve antigenic challenge. It is understood that the present methods provide an alternative means to achieve the goal of antigenic challenge and can be used separately or in conjunction with a challenge to determine efficacy or sufficiency.

Throughout this application the term “sufficient immune response” is used to describe an immune response of a large enough magnitude to provide an acceptable immune protection to the subject against future antigen encounter. It is understood that immune protection does not necessarily mean prevention of future antigenic encounter (e.g., infection), nor is it limited to a lack of any pathogenic symptoms. “Immune protection” means a prevention of the full onset of a pathogenic condition. Thus in one embodiment a “sufficient immune response” is a response that reduces the symptoms, magnitude, or duration of an infection or other disease condition when compared with an appropriate control. The control can be a subject that is exposed to an antigen before or without a sufficient immune response.

It is understood herein that an “immune response” refers to any inflammatory, humoral, or cell-mediated response that occurs for the purpose of eliminating an antigen. Such responses can include, but are not limited to, antibody production, cytokine secretion, complement activity, and cytolytic activity. In one embodiment, the immune response is a cytotoxic T lymphocyte (CTL) response. In another embodiment the immune response comprises the secretion of cytokines such as IFN-γ and TNF-α.

Thus in one embodiment, the present invention comprises a method of detecting a CTL response to a foreign antigen comprising introducing the antigen to a subject, collecting a sample from the subject, and detecting the presence of VLA+ (positive) antigen-specific T-cells in the sample (including, for example, a non-lymphoid peripheral tissue).

By “subject” is meant an individual. Preferably, the subject is a mammal such as a primate, and, more preferably, a human. The term “subject” can include domesticated animals, such as cats, dogs, etc., livestock (e.g., cattle, horses, pigs, sheep, goats, etc.), and laboratory animals (e.g., mouse, rabbit, rat, guinea pig, etc.).

“Antigen” means any native or foreign substance that is capable of eliciting an immune response. Preferably, the antigen will elicit a T-cell response. More preferably, the antigen will elicit a CD8+ T cell response. Such antigens can include but are not limited to peptides and/or proteins from a subject, virus, bacteria, yeast, or parasite. Antigens can also include vaccines (e.g., peptides, proteins, killed pathogens, or attenuated pathogens administered in a pharmaceutically acceptable carrier either prophylactically or therapeutically), and native peptides, polypeptides, and proteins.

It is understood that the antigen can be a viral antigen. Viral antigens can include any peptide, polypeptide, or protein from a virus. Thus in one embodiment the antigen can be an antigen from a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In particular, it is understood that the viral antigen can be an antigen from Influenza-A. Therefore it is understood that the present methods include methods of assessing the efficacy or sufficiency of an immune response to an Influenza-A antigen. Preferably the Influenza-A antigen is an attenuated or killed strain of Influenza-A.

Also disclosed are methods wherein the antigen is a bacterial antigen. The antigen, for example, can be a peptide, polypeptide, or protein selected from the group of bacteria consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Also disclosed are methods wherein the antigen is a fungal antigen. The antigen can be, for example, a peptide, polypeptide, or protein selected from the group of fungi consisting of Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata.

Also disclosed are methods wherein the antigen is a parasite antigen. The antigen can be, for example, a peptide, polypeptide, or protein selected from the group of parasitic organisms consisting of Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica.

Also disclosed are methods wherein the antigen is a cancer-related antigen. The antigen can be, for example, a peptide, polypeptide, or protein selected from the group of cancers consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumors, myelomas, AIDS-related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer.

The present methods can also be used to the efficacy of immune responses to an antigen related to an autoimmune or inflammatory condition. Such conditions include but are not limited to asthma, rheumatoid arthritis, reactive arthritis, spondylarthritis, systemic vasculitis, insulin dependent diabetes mellitus, multiple sclerosis, experimental allergic encephalomyelitis, Sjögren's syndrome, graft versus host disease, inflammatory bowel disease including Crohn's disease, ulcerative colitis, ischemia reperfasion injury, myocardial infarction, Alzheimer's disease, transplant rejection (allogeneic and xenogeneic), thermal trauma, any immune complex-induced inflammation, glomerulonephritis, myasthenia gravis, cerebral lupus, Guillaine-Barre syndrome, vasculitis, systemic sclerosis, anaphylaxis, catheter reactions, atheroma, infertility, thyroiditis, ARDS, post-bypass syndrome, hemodialysis, juvenile rheumatoid, Behcets syndrome, hemolytic anemia, pemphigus, bulbous pemphigoid, stroke, atherosclerosis, and scleroderma. In particular, the antigen can comprise an amyloid antigen (e.g., amyloid β peptide) thus providing an assessment of an immune response to Alzheimer's disease.

The methods disclosed herein comprise assessing the efficacy or sufficiency of an immune response to a selected antigen in a subject. The disclosed methods utilize tissue samples from the subject to provide the basis for assessment. Such tissue samples can include, but are not limited to, blood (including peripheral blood and peripheral blood mononuclear cells), tissue biopsy samples (e.g., spleen, liver, bone marrow, thymus, lung, kidney, brain, salivary glands, skin, lymph nodes, and intestinal tract), and specimens acquired by pulmonary lavage (e.g., bronchoalveolar lavage (BAL)). Thus it is understood that the tissue sample can be from both lymphoid and non-lymphoid tissue. Examples of non-lymphoid tissue include but are not limited to lung, liver, kidney, and gut. Lymphoid tissue includes both primary and secondary lymphoid organs such as the spleen, bone marrow, thymus, and lymph nodes.

Various methods can be used to determine the presence of antigen-specific T cells that are VLA-1+. Such methods include but are not limited to immunohistochemistry, intracellular cytokine staining, cytokine ELISpot assays, and flow cytometric staining using antigen-specific dimer staining, and tetramer staining. Cytokine assays employed to identify the antigen-specific T cells utilize peptides corresponding to known immunoresponsive epitopes to stimulate cytokine secretion. In the case of intracellular staining, antigen-specific cells are visualized on a flow cytometer by using any one of the many intracellular staining protocols known in the art to identify those cells that, upon peptide stimulation secrete a cytokine. This staining can be accomplished in conjunction with surface staining for VLA-1 (CD49a) using an anti-CD49a antibody. Double positive cells (i.e., those cells that express CD49a on their surface and secrete the cytokine) can be used to assess efficacy or a sufficiency of the immune response.

A variety of markers for effector and memory T cells can be used. For example, the disclosed methods can comprise identifying VLA+, antigen-specific T-cells that are also CD62L^{lo}, CD69+, CD43+, CD27+, CD44+, CD45RO+, and/or CD45RA− (negative). It is understood that any combination of one or more of CD62L, CD69, CD43, CD27, CD44, CD45RO, and CD45RA can be used in addition to VLA and an antigen-specific marker (e.g., tetramer, dimer, intracellular cytokine staining).

Similarly, tetramers or dimers can be used to identify antigen-specific T-cells. Briefly, the tetramer is four MHC molecules with bound peptides corresponding to antigenic epitopes. The tetramer is so called because the reagent comprises four MHC-peptide molecules bound to a common reagent. Dimers refer to the same technology employing only two MHC-peptide constructs instead of four as in the tetramer. T cells specific for the peptide of the tetramer or dimmer, will bind the tetramer and, as these reagents are labeled, the cells binding to them will become labeled. The purpose of the multiple binding sites is to increase the avidity, time of binding as well as the opportunity for binding. Thus, optionally, the number of tetramer positive, VLA-1+ positive cells in a tissue sample effective or sufficient is assessed, wherein a number above a suitable control is an efficacious immune response.

The disclosed method of assessing the efficacy or sufficiency of an immune response to a selected antigen in a subject is related to identifying the peripheral effector and memory cells. Effector T cells are antigen-specific T cells that provide the majority of the cell-mediated response in an acute infection or in the initial control of a chronic infection. Memory T cells are antigenic specific T cells that can be maintained for the life-span of the host without further antigenic stimulation and can confer immunological protection to the subject against the antigen. Memory cells can be subdivided into two populations of cells, central and effector memory cells (also referred to as peripheral memory T cells), largely based on their localization. Central memory cells typically reside in secondary lymphoid organs whereas effector (peripheral memory T cells) memory cells typical reside in the periphery. It is the central memory population of T cells that can be maintained for the life of the host without further antigenic stimulation. In the effector memory T cell population, VLA-1+ (positive) cells are maintained for extended periods of time and provide the first response to localized infections or systemic infections that are initiated in the periphery.

Antigen specific T cells are formed in response to an antigenic insult and antigen-specific T cells can be observed between 4 and 8 days post antigenic experience. Initially, the effector T cell population is the dominant T cell population, but between 8 and 15 days post antigenic experience, 80-90% of the effector T cells are lost to apoptosis. During this time, memory T cells become the dominant T cell population and comprise over 90% of the antigen-specific T-cells by 30 days post antigenic experience. Thus, tissue samples containing antigen specific T cells may be collected 6-10 days after the antigen introduction. Tissue samples containing T cells may be collected between 10 and 14 days. Tissue samples may also be collected 14-21 days after antigen introduction. Also disclosed are methods wherein tissue samples are collected 21-30 days after antigen introduction. Also disclosed are methods, wherein the tissue sample is collected 30-60 days after the antigen introduction. Also disclosed are methods, wherein the tissue sample is collected 2-6 months after the antigen introduction. Also disclosed are methods, wherein the tissue sample is collected 6 months to 1 year after the antigen introduction. Also disclosed are methods, wherein the tissue sample is collected 10 years after the antigen introduction.

A method of screening for an antigen that elicits a sufficient immune response in a subject comprising introducing into the subject the antigen to be tested, collecting a tissue sample from the subject, and measuring VLA-1+ (positive), antigen-specific T-cells in the sample, a high level of VLA-1+ (positive) antigen-specific T-cells as compared to a control sample indicating an antigen that elicits an immune response in the subject.

“Quantifying” means the act of placing a numerical or qualitative value on the magnitude of the item being measured. For example, the number VLA-1+ antigen-specific cells can be quantified by determining by flow cytometry the percentage that the VLA-1+ antigen-specific cells represent in a tissue sample and multiplying that percentage by the total number of cells in the tissue or volume of tissue.

“High level” means the magnitude of positively identified cells that can be identified as being greater than the magnitude of a control sample. A large magnitude above the control (i.e., a higher the level) indicates a larger immune response and therefore a response more likely to confer immunological protection. For example, levels of VLA-1 expression on the antigen-specific cells in the blood that are greater than 25%, or in the tissues that are greater than 60% would be expected to correlate with improved protection.

Methods of Treatment

The methods disclosed herein include methods of treating a subject with a disease comprising administering to the subject an antigen identified by the screening methods disclosed herein, wherein the antigen is related to the disease to be treated. For example, one embodiment of the present application is a method of treating a subject with a disease comprising administering to the subject a selected antigen identified by introducing into the subject the antigen to be tested, collecting a tissue sample from the subject, and measuring VLA-1+ (positive), antigen-specific T-cells in the sample, a high level of VLA-1+ (positive) antigen-specific T-cells as compared to a control sample indicating an antigen that elicits an immune response in the subject and an antigen useful in treating the disease. Furthermore, the disclosed methods include the adoptive transfer of VLA+ antigen-specific T-cells from a donor to a subject, wherein the transferred cells confer immunological protection to the subject.

Also disclosed are methods of treating a subject with a disease comprising administering to a subject VLA-1+ (positive), antigen-specific T-cells isolated from a donor subject. The VLA-1+ (positive), antigen-specific T-cells are isolated from a subject by introducing into the subject a selected antigen, collecting a tissue sample from the subject, and isolating VLA-1+ (positive), antigen-specific T-cells from the sample. Such isolated cells can be administered to the subject to be treated. Also disclosed are methods of isolating VLA-1+ (positive), antigen-specific T-cells comprising isolating from a donor subject VLA-1+(positive), antigen-specific T-cells comprising introducing into the subject a selected antigen, collecting a tissue sample from the subject, and isolating VLA-1+ (positive), antigen-specific T-cells from the sample.

The methods of treating a subject disclosed herein comprise the administering of VLA+, antigen-specific T-cells or a selected antigen to a subject. It is understood that it may be necessary to provide a suitable carrier for the cells or a selected antigen so the composition may be safely administered to the subject. Thus, herein specifically contemplated are pharmaceutical compositions comprising the antigen-specific, VLA+ cells or antigen of the invention.

“Donor” means a source of a peptide, protein, cell, tissue, or organ to be used in the methods described herein, wherein the source is not the subject to be treated.

“Treatment” or “treating” means to administer a composition to a subject with an undesired condition or at risk for the condition. The condition can be any pathogenic disease, autoimmune disease, cancer or inflammatory condition. The effect of the administration of the composition to the subject can have the effect of but is not limited to reducing the symptoms or duration of the condition, a reduction in the severity of the condition, or the complete ablation of the condition.

By “effective amount” is meant a therapeutic amount needed to achieve the desired result or results, e.g., establishing an immune response that can confer immunological protection to the subject. It is understood that immunological protection includes but is not limited to prevention of subsequent infections; reduction of the effects or symptoms of subsequent infections or conditions; reduction in the duration of the infection or condition; lessening of severity of a disease or condition; or reduced antigenic load relative to non-treated controls.

The disclosed methods can be used to treat any infectious disease, as well as, autoimmune and inflammatory diseases and cancers. It is understood and herein contemplated that any VLA-1+ (positive) antigen-specific T cell may be used in the disclosed treatment methods. Such T cells include but are not limited to effector t-cells, central memory T-cells, and peripheral (also referred to as effector) memory T-cells. For example, the VLA-1+ D^{b}NP-specific peripheral memory T cells can be transferred from a donor to the subject to treat for Influenza. Also, for example, an antigen that stimulates a high level of VLA-1+ D^{b}NP-specific peripheral memory T cells can be administered to a subject to treat a disease or condition such as Influenza.

It is understood that in one embodiment the disease can be a viral infection. Thus the disease can be caused by a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian immunodeficiency virus, Human Immunodeficiency virus type-1, and Human Immunodeficiency virus type-2.

In particular, the virus can be Influenza-A. Thus the present methods include methods of treating a subject with Influenza-A comprising administering to the subject Influenza-A-specific, VLA+, T-cells from a donor source or administering to the subject an antigen identified by the screening method described herein.

Also disclosed are methods wherein the disease is a bacterial infection. Thus, specifically contemplated are methods treating a disease in a subject, wherein the disease is a bacterial infection selected from the group of bacteria consisting of M. tuberculosis, M. bovis, M. bovis strain BCG, BCG substrains, M. avium, M. intracellulare, M. africanum, M. kansasii, M. marinum, M. ulcerans, M avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria rumzinantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Also disclosed are methods wherein the disease is a fungal infection. Fungal species and their proteins are well-known in the art. Thus, specifically contemplated are methods treating a disease in a subject, wherein the disease is a fungal infection selected from the group of bacteria consisting of Candida albicans, Cryptococcus neoformans, Histoplama capsulatum, Aspergillus fumigatus, Coccidiodes immitis, Paracoccidiodes brasiliensis, Blastomyces derimzitidis, Pneomocystis carnii, Penicillium marnieffi, and Alternaria alternata.

Also disclosed are methods wherein the disease is a parasitic infection. Thus, specifically contemplated are methods treating a disease in a subject, wherein the disease is a parasitic infection selected from the group of bacteria consisting of Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica.

Also disclosed are methods wherein the disease is a cancer. Thus, specifically contemplated are methods treating a disease in a subject, wherein the disease is a cancer selected from the group of bacteria consisting of from the selected from the group of cancers consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumors, myelomas, AIDS-related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer.

The present methods can also be used to treat autoimmune or inflammatory conditions. Such conditions include but are not limited to asthma, systemic lupus erythematosus, rheumatoid arthritis, reactive arthritis, spondylarthritis, systemic vasculitis, insulin dependent diabetes mellitus, multiple sclerosis, experimental allergic encephalomyelitis, Sjögren's syndrome, graft versus host disease, inflammatory bowel disease including Crohn's disease, ulcerative colitis, ischemia reperfusion injury, myocardial infarction, Alzheimer's disease, transplant rejection (allogeneic and xenogeneic), thermal trauma, any immune complex-induced inflammation, glomerulonephritis, myasthenia gravis, cerebral lupus, Guillaine-Barre syndrome, vasculitis, systemic sclerosis, anaphylaxis, catheter reactions, atheroma, infertility, thyroiditis, ARDS, post-bypass syndrome, hemodialysis, juvenile rheumatoid, Behcets syndrome, hemolytic anemia, pemphigus, bulbous pemphigoid, stroke, atherosclerosis, and scleroderma. In particular, the antigen can comprise an amyloid antigen (e.g., amyloid P peptide) thus providing an assessment of an immune response to Alzheimer's disease.

Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of subjects is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the disease or disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The compositions, can be used therapeutically in combination with a pharmaceutically acceptable carrier. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

### EXAMPLE

### The Collagen-Binding α1β1 Integrin VLA-1 Regulates CD8 T Cell-Mediated Immune Protection against Heterologous Influenza Infection

In the mouse, experimental infection of C57BL/6 mice with serologically distinct influenza H3N2 A/HK/x31 (X31) and H1N1 A/PR8 (PR8) viruses is a well characterized model of heterosubtypic immunity (Bennink, J., et al. (1978) Immunology 35, 503-509; Effros, R. B., et al. (1978) Cell Immunol 36, 345-353; Effros, R. B., et al. (1977) J Exp Med 145, 557-568; Liang, S., et al. (1994) J Immunol 152, 1653-1661; Yewdell, J. W., et al. (1985) Proc Natl Acad Sci USA 82, 1785-1789). In most strains of mice, X31 produces a fairly benign infection, with peak cellular infiltration of the lung at day 8, clearance of the virus by day 9, and rarely produces death even when a high inoculum dose (105 EID₅₀) is administered (Allan, W., et al. (1990) J Immunol 144, 3980-3986). PR8 infection, on the other hand, causes death in the majority of infected B6 mice, even when low doses of virus (10² EID₅₀) are used (Ennis, F. A., et al. (1976) Dev Biol Stand 33, 220-225). The internal nucleoprotein (NP) is shared between the two viruses (Tite, J. P., et al. (1990) Immunology 71, 202-207) and prior immunization with X31 elicits NP reactive CD8 cytotoxic T lymphocytes (CTL), specific for an H2-D^{b}-restricted epitope of the internal nucleoprotein (NP₃₆₆₋₃₇₄) (SEQ ID NO: 1), that can protect mice from PR8 challenge (Bennink, J., et al. (1978) Immunology 35, 503-509; Effros, R. B., et al. (1978) Cell Immunol 36, 345-353; Effros, R. B., et al. (1977) J Exp Med 145, 557-568). Yet this heterosubtypic protective immunity can wane with time (Liang, S., et al. (1994) J Immunol 152, 1653-1661), a phenomenon related to the number of flu-specific CD8 T cells in the lung (Hogan, R. J., et al. (2001) J Immunol 166, 1813-1822).

This latter issue is relevant to understanding the persistence of T cell memory and protection. CD8 memory T cells against viral antigens persist indefinitely in secondary lymphoid organs (Doherty, P. C., et al. (1997) Semin Immunol 9, 365-373; Doherty, P. C., et al. (1996). Immunol Rev 150, 23-44). However, a number of reports have demonstrated that, in spite of substantial numbers of memory CD8 T cells in the spleen and lymph nodes, protection against infection in non-lymphoid organs is short lived (Bachmann, M. F., et al. (1997) Proc Natl Acad Sci USA 94, 640-645; Hogan, R. J., et al. (2001) J Immunol 166, 1813-1822; Liang, S., et al. (1994) J Immunol 152, 1653-1661). In the flu system, CD8 T cell mediated protective heterosubtypic immunity in the upper and lower respiratory tract of BALB/c mice is short lived, disappearing 4 to 6 months after priming (Liang, S., et al. (1994) J Immunol 152, 1653-1661). Though there were many flu-specific CD8 T cells in the spleen, this protection was later shown to depend on the number of virus-specific CD8 T cells resident in the lung (Hogan, R. J., et al. (2001) J Immunol 166, 1813-1822). A similar phenomenon discovered using the LCMV model suggested the loss of peripheral immune protection was related to the activation status of the memory CD8 T cells (Bachmann, M. F., et al. (1997) Proc Natl Acad Sci USA 94, 640-645). In this model, despite long lasting elevated precursor frequencies to LCMV in the “central” lymphoid tissue, immune protection against infection in “peripheral” (non-lymphoid) organs was short-lived (Bachmann, M. F., et al. (1997) Proc Natl Acad Sci USA 94, 640-645). It was suggested that there is a time-dependent loss of effector CTL and that persisting antigen is essential to keep CTL poised to act against emerging infections (Bachmann, M. F., et al. (1997) Proc Natl Acad Sci USA 94, 640-645). Taken together these studies show that efficient secondary immune protection depends on the presence of activated effector/memory T cells in the peripheral tissues.

One of the major T cell surface receptors for collagen is the α1β1 integrin heterodimer, originally designated as Very Late Antigen (VLA)-1 (Hemler, M. E., et al. (1986) J of Clin Invest 78, 696-702). VLA-1 primarily binds to Type V, but also Type I collagen (Belkin, V. M., et al. (1990) J. Cell Bio. 111, 2159-2170; Hemler, M. E. (1990) Ann Rev of Imm. 8, 365-400). Analysis of the expression of al (CD49a) integrin expression on cells from secondary lymphoid organs reveals little detectable expression on resting peripheral T cells (Bank, I., et al. (1991) J. Clin Imm. 11, 29-38; Goldman, R., et al. (1992) European J. of Imm. 22, 1109-1114; Hemler, M. E. (1990) Ann Rev of Imm. 8, 365-400; Stemme, S., et al. (1992) Arteriosclerosis & Thrombosis 12, 206-211). While the role of VLA-1 on T cells in disease is not known, infiltrating T cells have been observed in vivo to express VLA-1 in the synovium of rheumatoid arthritis (Hemler, M. E., et al. (1986) J of Clin Invest 78, 696-702; Takahashi, et al. (1992) European J of Immunol 22, 2879-2885) and in atherosclerotic plaques (Stemme, S., et al. (1992) Arteriosclerosis & Thrombosis 12, 206-211). Treatment of mice with hamster mAbs that block VLA-1 (Ha 31/8) (Mendrick, D. L., et al. (1995) Lab Invest 72, 367-375) abrogate the induction of both experimental rheumatoid arthritis and delayed type hypersensitivity (DTH) responses (de Fougerolles, A. R., et al. (2000) J Clin Invest 105, 721-729). No evidence for functional deletion of effector cells was apparent, since treated mice responded normally when the blocking antibody treatment was suspended (de Fougerolles, A. R., et al. (2000) J Clin Invest 105, 721-729). Similarly, α1 deficient mice were resistant to the induction of arthritis and hypersensitivity reactions (de Fougerolles, A. R., et al. (2000) J Clin Invest 105, 721-729), supporting that the mode of action for the mAb inhibition studies is related to diminished VLA-1 function. Both the DTH and arthritis models are examples of inflammation driven by CD4 T cells (Hernandez-Pando, R., and Rook, G. A. (1994) Immunology 82, 591-595; Seki, N., et al. (1989) Reg Immunol 2, 203-212), making a strong case for the role of VLA-1 in inflammatory CD4 T cell biology. In the human lung, VLA-1 positive T cells have been found compartmentalized on the epithelial surface of the lower respiratory tract (Saltini, C., et al. (1988) Clin Immunol Immunopathol 46, 221-233), and these lung T cells appeared to exchange very slowly with the systemic lymphoid organs (Saltini, C., et al. (1988) Clin Immunol Immunopathol 46, 221-233).

VLA-1 is Expressed by the Majority of Peripheral CD8 T Cells during Influenza Virus Infection:

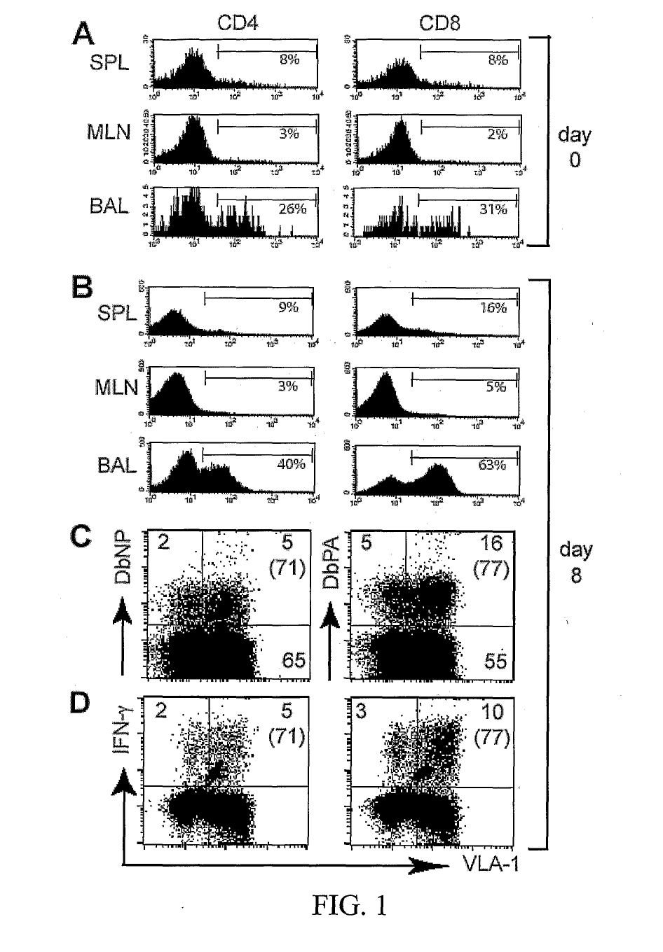

Examination of VLA-1 expression in lymphoid organs of naïve 8 week old C57BL/6 mice reveals minor populations of CD4 and CD8 T cells that express VLA-1 (FIG. 1A, day j). Interestingly, even in these resting animals, though few lymphocytes can be isolated from the lung by bronchoalveolar lavage (BAL), a substantial proportion of the CD4 (26%) and CD8 (31%) T cells express VLA-1 (FIG. 1A), much greater than that seen in the draining mediastinal lymph node (MLN, 2-3%) or the spleen (8%).

Strikingly, respiratory infection with influenza X31 causes a substantial increase in the numbers of both CD4 and CD8 T cells that express VLA-1 (FIG. 1B). The cellular immune response to influenza peaks 8 days after inoculation (Allan, W., et al. (1990) J Immunol 144, 3980-3986), and at this time the majority (63%) of CD8 T cells in the lung express VLA-1. CD4 T cells in the BAL also express VLA-1 (40%). Again, the proportion of CD4 or CD8 T cells expressing VLA-1 in the MLN and spleen is modest at 3-5% in the MLN and 9-16% in the spleen. These observations show this Very Late Antigen (Hemler, M. E., et al. (1985) Eur J Immunol 15, 502-508) is expressed early during virus infection in an organ-selective manner, suggesting that some important signal is present in this context that was missing from the in vitro cultures where VLA-1 was originally described (Hemler, M. E., and Jacobson, J. G. (1987) J Immunol 138, 2941-2948).

Expression of VLA-1 on Flu Specific CD8 T Cells during the Infection:

There is very little non-specific recruitment of CD8 T cells to the lung during influenza infection (Topham, D. J., et al. (2001) J Immunol 167, 6983-6990). Since VLA-1 was expressed by most of the CD8 T cells in the lung, it was likely that many of these were influenza specific. To investigate this possibility, CD8 T cells from the BAL, lung tissue, MLN, and spleen were stained with H2-D^{b} class I MHC tetramers containing the immunodominant nucleoprotein NP366-374 and acid polymerase (PA₂₂₄₋₂₃₃) (SEQ ID NO: 2) peptide epitopes (Belz, G. T., et al. (2000) J Virol 74, 3486-3493; Flynn, K. J., et al. (1998) Immunity 8, 683-691). At the peak of the acute primary immune response (d8), 71 and 77%, respectively, of the NP or PA tetramer+ CD8 T cells in the BAL also expressed VLA-1 (FIG. 1C). These proportions are similar to those seen for the CD8 population as a whole. The data make it clear that, even among CD8 T cells with identifiable virus-specificity, there are two subpopulations defined by VLA-1 integrin expression.

The differences between the VLA-1+ and VLA-1− CD8 populations in the lung were not known. Certainly, the presence of CD8 T cells in the lung lacking VLA-1 expression indicated that this integrin was not essential for CD8 T cells recruitment. Alternatively, expression might be acquired in the lung environment. On the other hand, expression of VLA-1 on some flu specific CD8 T cells in the MLN and spleen indicates that some CD8 cells can express VLA-1 outside of the infected lung.

To determine whether VLA-1 integrin expression correlated with effector function, BAL CD8 T cells were stimulated with NP₃₆₆₋₃₇₄ or PA₂₂₄₋₂₃₃ in the presence of Brefeldin A, and then analyzed for intracellular IFN-γ (Belz, G. T., et al. (2000) J Virol 74, 3486-3493; Flynn, K. J., et al. (1998) Immunity 8, 683-691). Of the CD8 T cells that produced IFN-γ, roughly 80% were also positive for VLA-1 (FIG. 1D).

Virus-Specific VLA-1-Expressing CD8 T Cells Accumulate in the Lung During Resolution of the Infection:

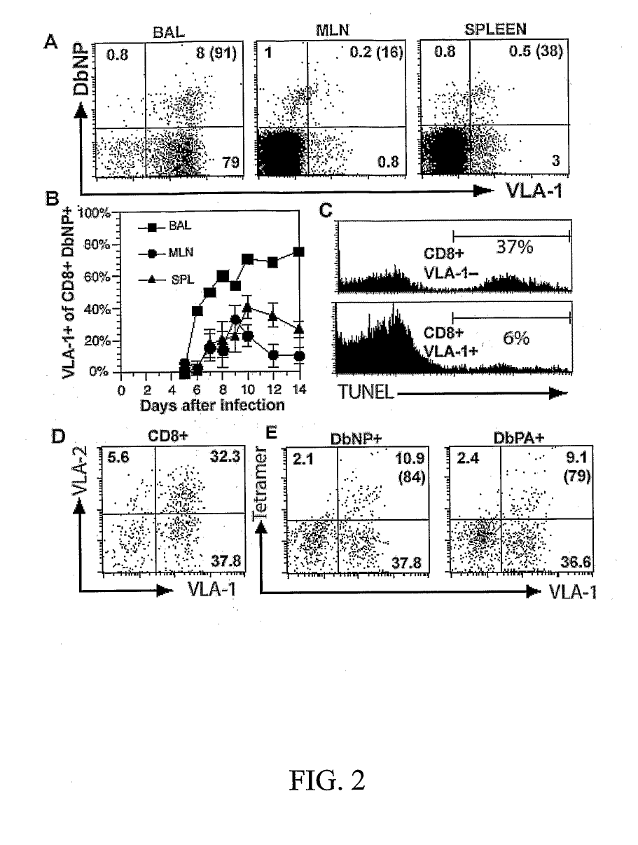

Infection with influenza virus results in permanent increases in lymphocyte numbers in the lung (Doherty, P. C., et al. (1997) Semin Immunol 9, 365-373). During the initial phase (d0-8) of a primary immune response to flu, the number of CD8 T cells in the lung increases by three orders of magnitude. Replication of the virus is controlled within 9 days of inoculation in B6 mice, and the inflammatory response resolves (Topham, D. J., and Doherty, P. C. (1998) J Virol 72, 882-885). During this resolution phase, the number of CD8 T cells in the lung drops by one to two orders of magnitude (Tripp, R. A., et al. (1995). J Immunol 154, 6013-6021), but does not return to pre-infection levels. Many of the CD8 T cells infiltrating the lung during the acute phase undergo deletion by apoptosis as the infection resolves (Belz, G. T., et al. (1998) Proc Natl Acad Sci USA 95, 13812-13817), presumably due to diminishing viral antigens. However, a few flu-specific CD8 T cells remain in the lung for extended periods of time (Hogan, R. J., et al. (2001) J Immunol 166, 1813-1822). Analysis of VLA-1 expression on the D^{b}/NP+ CD8 T cells 14 days after infection (FIG. 2A) revealed that the cells remaining in the lung were predominantly VLA-1+, while those in the MLN were mostly VLA-1−, and those in the spleen and blood were mixed. This was the first indication that VLA-1 might affect the distribution of virus-specific CD8 T cells, with the VLA-1+ population favoring non-lymphoid tissues.

To determine how this affected the flu specific CD8 T cell population, CD8+ T cells were co-stained with either D^{b}NP or D^{b}PA tetramers and Ha31/8 antibody to α1. Analysis of α1 expression on the tetramer+ cells in the lung showed that these CD8 T cells selectively increased in proportion as the infection resolved (FIG. 2B). The increase in the proportion of flu-specific VLA-1+ CD8 T cells after clearance of virus was not maintained in the MLN or spleen. The data are particularly striking considering that this is the period of time when the total number of lymphocytes in the lung is exponentially dropping.

Reduced Apoptosis among the VLA-1+CD8 T Cells in the Lung:

As the number of CD8 T cells in the lung drops during resolution of flu infection, most of these cells die by apoptosis (Belz, G. T., et al. (1998) Proc Natl Acad Sci USA 95, 13812-13817). Though many of the apoptotic cells are rapidly cleared from lung and some accumulate in the liver (Belz, G. T., et al. (1998) Proc Natl Acad Sci USA 95, 13812-13817), a substantial number of cells probably die in the lung and are cleared by alveolar macrophages. To determine whether the VLA-1-expressing population might have some survival advantage, the TUNEL assay (Darzynkiewicz, Z., et al. (1992) Cytometry 13, 795-808) was used to estimate the proportion of CD8 T cells undergoing apoptosis.

At 8 days after infection, the cellular immune response is at its peak, and virus is being cleared rapidly. This rapid loss of antigen may drive the passive apoptosis of the virus-specific CD8 T cells (Van Parijs, L., and Abbas, A. K. (1998) Science 280, 243-248). At this time point, the VLA-1+ population of CD8 T cells showed reduced apoptosis compared to the VLA-1− subset (FIG. 2C). This is in direct contrast to a report that α2β1 integrin (VLA-2) expressing CD8 T cells were more likely to apoptose in the context of influenza infection (Kambayashi, T., et al. (2001) Eur J Immunol 31, 1523-1530). The data presented herein demonstrate that the VLA-1+ CD8+ T cells in the lung have a unique survival advantage, related to signals delivered through VLA-1.

Reduced apoptosis among the VLA-1+ CD8 T cells may partially explain how this population becomes predominant as the infection is resolved. Increased survival and the adhesive functions of VLA-1, which would be predicted to localize T cells to areas of collagen, combine to create a model in which VLA-1 is instrumental in the establishment of memory T cells in non-lymphoid tissues.

Persistence of VLA-1+ CD8 T Cells in Recovered Animals and Proximity to Collagen:

Approximately 2 months after infection with influenza, analysis of CD8 T cells recoverable by BAL revealed a significant population of CD8 T cells in the lung that expressed VLA-1 (FIG. 2D). Further analysis of the CD8 T cells in the lung by staining with D^{b}NP or D^{b}PA tetramers showed that 79-84% of D^{b}NP and D^{b}PA tetramer positive CD8 T cells were VLA-1⁺ (FIG. 2E).

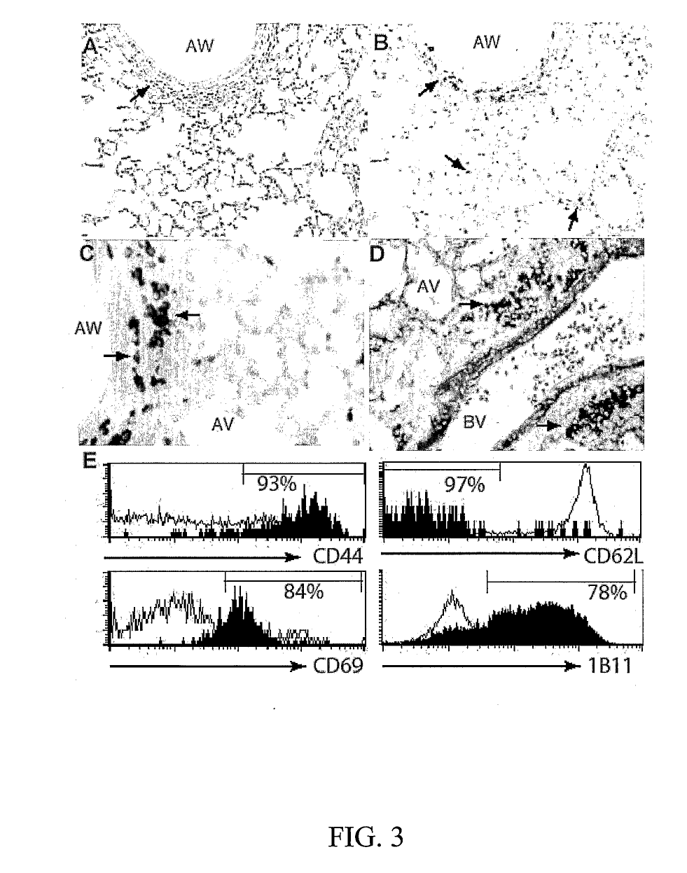

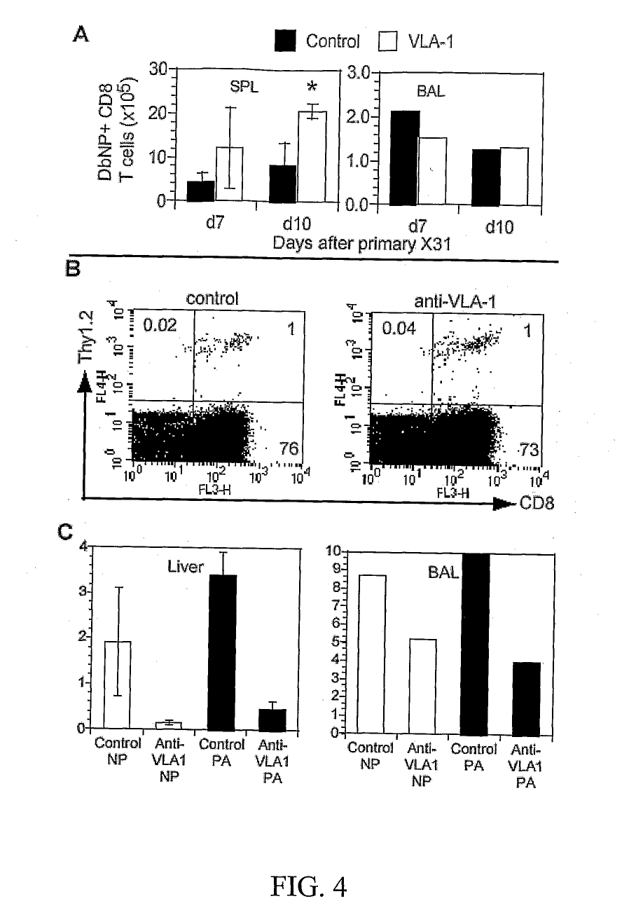

Together, the data indicate that as the infection resolves, there is a selective retention of VLA-1+ CD8 T cells in the lung. The high proportion of CD8 T cells in the lung that express VLA-1, and the more limited distribution of VLA-1 on T cells in the secondary lymphoid organs, indicated that it was functioning to localize the CD8 CTL within areas of accessible collagen in the lung tissue. To investigate this phenomenon, lungs of influenza immune mice were processed for immunohistochemistry. Formalin- or zinc-fixed serial tissue sections from day 30 flu-immune mice were stained with the trichrome stain to identify collagen (FIGS. 3A, D), H & E (FIG. 3B) or anti-CD813 (FIGS. 3C, D). CD3+ and CD8+ cells could be seen in the interstitium adjacent to conducting airways and vasculature, where trichrome staining was evident, indicating a close association with collagen. Dual anti-CD8β and trichrome staining was performed to help assess the relationship between the CD8+ cells and the collagen deposits. CD8+ cells were clearly localized within the interstitial areas surrounding the airways and major blood vessels (FIG. 4D), where collagen content is greatest as seen in the trichrome stain. This staining pattern fits with the expected distribution of Type I and IV collagen (the primary substrates for VLA-1), which constitute important structural components of the perivascular and peribronchial interstitium (Type I) as well as the basement membranes (Type IV) of vascular endothelium and airway epithelium (Miner, J. H., and Sanes, J. R. (1994) J Cell Bio. 127, 879-891; Sado, Y., et al. (1998) J. Biochem. 123, 767-776).

Activation Profiles of VLA-1+CD8 T Cells:

Previous studies describing the presence of virus-specific CD8 T cells in non-lymphoid tissues showed that these T cells had an activated profile usually associated with effector/memory cells (Masopust, D., et al. (2001) Science 291, 2413-2417). To investigate whether the VLA-1+ population of CD8 T cells matched this effector/memory T cell profile, 30 days after X31 infection, the VLA-1+ CD8+ T cells were analyzed for the activation markers CD69, CD25, CD44, 1B11, and CD62L. The majority of the VLA-1+ CD8+ T cells in the lung were CD69+, CD25−, CD44high, 1B11+ and CD62L low (FIG. 4E), including the D^{b}NP and D^{b}PA tetramer positive subsets. This phenotype indicates that these T cells were poised for mounting rapid effector functions should the pathogen be re-encountered. Thus they could be important in secondary immunity.

VLA-1 is not Essential for Recruitment of CD8 T Cells to the Lung During Primary Infection:

To investigate whether VLA-1 integrin participated in the process of T cell recruitment to the lung, animals were treated with a monoclonal antibody against the α1 subunit (Ha31/8) of VLA-1, which blocks binding to collagen (Mendrick, D. L., et al. (1995) Lab Invest 72, 367-375), or a control hamster Ig (Ha4/8). Each animal received 250 μg of each affinity purified, endotoxin-free antibody every other day beginning prior to inoculation with virus, and continuing on alternate days throughout the course of the experiment. No reproducible effect was seen on the numbers of CD8 T cells or D^{b}NP tetramer positive CD8 T cells in the lung (FIG. 4A). D^{b}NP+CD8 T cells in the MLN were also unaffected, but they were significantly (p<0.01) increased in the spleen at day 10 (FIG. 4A), indicating redistribution had occurred. Viral clearance was similar in both groups. The high expression of VLA-1 on the virus-specific CD8 T cells during the infection, the absence of an effect of anti-al on viral clearance, and minimal changes in cell distributions show that the Ha31/8 mAb does not deplete the antiviral T cells, and is in line with published reports utilizing the same antibodies (de Fougerolles, A. R., et al. (2000) J Clin Invest 105, 721-729). The data also show that inhibition of VLA-1 did not inhibit primary recruitment of T cells to the lung.

To further test whether VLA-1 integrin was involved in the recruitment of activated CD8 T cells to the infected lung, the following adoptive transfer study was performed. Lung lymphocytes from day 8 X31 flu infected Thy-1.2+ B6 mice were collected by bronchoalveolar lavage and stained with either an isotype control hamster Ig (Ha 4/8) or the anti-VLA-1 hamster antibody (Ha31/8). Flow cytometric analysis of these cells prior to transfer revealed that 56% were CD8+, and 75% of the CD8 cells also expressed VLA-1. The cells were then adoptively transferred by i.v. injection into day 6 X31 infected Thy1.1+ B6.PL congenic hosts. The hosts were also treated with 250 μg of the same anti-α1 or control hamster mAb. Twenty-four hours later, the BAL was collected and analyzed for the presence of Thy1.2+ donor CD8 T cells (FIG. 4B). The number and proportion of Thy1.2+ CD8 T cells in the BAL was identical in both the control and anti-α1 treated groups. It was concluded that VLA-1 does not participate in the recruitment of CD8 T cells to the infected lung and that the anti-α1 Ha 31/8 monoclonal antibody does not deplete the activated CD8 T cells.

Finally, in mice genetically deficient in α1 integrin (VLA-1 KO), primary infection with X31 revealed no increase in susceptibility to infection, or defect in the control of viral replication. The proportions of virus-specific CD8 cells in the lung were also similar to wild-type mice. This indicates that there are no defects in the ability to generate a protective primary influenza specific T cell response in the absence of VLA-1. These results strongly support those obtained using blocking antibodies and establish that VLA-1 does not have a major role in the recruitment of immune cells to the lung during infection.

Presence of VLA-1+ CD8 T Cells in Other Tissues and the Diaspora Effect:

An emerging paradigm in viral immunity is that the pathogen specific T cells can distribute though all the tissues of the body, even those that were not inflamed or involved in the primary infection (Masopust, D., et al. (2001) Science 291, 2413-2417; Reinhardt, R. L., et al. (2001) Nature 410, 101-105). For example, after an influenza infection, the flu-specific CD8 T cells undergo a “diaspora” effect that distributes them to many if not all organs (Doherty, P. C., et al. (2000) Philos Trans R Soc Lond B Biol Sci 355, 1093-1101; Marshall, D. R., et al. (2001) Proc Natl Acad Sci USA 98, 6313-6318). It is not known whether these cells come from the lung or secondary lymphoid organs. To see whether the appearance of flu specific VLA-1+ CD8 T cells was organ specific, affecting only the lung, several other non-lymphoid tissues were sampled. It was found that in most organs the majority (75-85%) of CD8 T cells isolated from non-lymphoid tissues expressed VLA-1, while in the lymphoid organs VLA-1+ CD8 T cells were a minority (Table 1).

TABLE 1
Predominance of VLA-1⁺ CD8⁺ T cells among non-lymphoid
organs compared to lymphoid organs

| | | | Of the | | Of the CD8⁺ |
|---|---|---|---|---|---|
| | | | CD8⁺ cellsⁱⁱⁱ | | D^{b}NP⁺ cells^{iv} |
| | | CD8⁺ | D^{b}NP⁺ | VLA-1⁺ | VLA-1⁺ |
| | Organⁱ | (%)ⁱⁱ | (%) | (%) | (%) |
| Non-lymphoid | BAL | 20.9 | 12.2 | 76.4 | 86.9 |
| | Lung | 44.6 | 11.1 | 80.7 | 94.2 |
| | Kidney | 3.8 | 33.1 | 74.4 | 94.3 |
| | Liver | 7.5 | 2.5 | 86.5 | 88.0 |
| | Salivary | 10.6 | 34.3 | 88.0 | 88.0 |
| | gland | | | | |
| Lymphoid | CLN | 23.0 | 0.4 | 0.4 | 31.8 |
| | MLN | 22.6 | 1.4 | 2.1 | 16.1 |
| | MesLN | 25.8 | 0.7 | 0.9 | 56.7 |
| | Spleen | 11.9 | 3.1 | 6.0 | 68.2 |
| | PBL | 24.6 | 0.7 | 4.0 | 16.9 |

ⁱC57BL/6 female mice were infected with influenza A X31 and allowed to recover from infection. 50-60 days after infection, lymphocytes were isolated from the indicated organs by excision, treatment with collagenase and DNAse, and disruption in a Dounce homogenizer.
ⁱⁱSingle cell suspensions were stained with anti-CD8-TC (CT8a) and the percent CD8+ cells within a lymphocyte gate determined by flow cytometric analysis.
ⁱⁱⁱSingle cell suspensions were stained with anti-CD8-TC (CT8a), anti-VLA1-Alexa488 (Ha31/8), and DbNP-PE tetramer. Cells were analyzed by flow cytometry after gating on lymphocytes and CD8+ events to determine the percent of CD8+ cells that were tetramer+ or VLA1+.
^{iv}After gating on lymphocytes that were positive for both CD8 and DbNP tetramer, the proportion of these that were VLA-1+ was determined.

When the flu specific CD8 T cells were identified with tetramers, the pattern persisted with 79-94% of the flu-specific CD8 T cells in the non-lymphoid organs expressing VLA-1 (FIG. 2E, Table 1). As has been described (Hogan, R. J., et al. (2001) J Immunol 166, 1813-1822), flu specific CD8+ T cells persist in the lung for several months, and herein were found to continue to express α1 integrin even 6 months after the primary infection.

The data support two conclusions: VLA-1 expression identified a population of CD8 T cells that is selective for non-lymphoid tissues; and secondly, since not all the CD8 T cells in the tissue were VLA-1+, that there were at least two subpopulations of CD8 T cells that migrate to non-lymphoid tissues.

Blocking VLA-1 in Immune Mice Reduces the Number of Memory CD8 T Cells in the Periphery:

The preferential accumulation of VLA-1+ NP-specific CD8 T cells led to the hypothesis that VLA-1 integrin mediated binding might be responsible for the retention of T cells in the lung and other non-lymphoid tissues. Since VLA-1 binds to Types I and IV collagen components of extracellular matrix (Hemler, M. E. (1990) Ann Rev of Imm. 8, 365-400), it could retain T cells within tissue via attachment to collagen. To test this hypothesis, influenza immune animals (>50 days after primary infection) were treated with anti-α1 (Ha 31/8), or control hamster Ig (Ha 4/8) mAb prior to harvesting lymphocyte populations. The lymphocytes were then stained for CD4 and CD8, as well as D^{b}NP and D^{b}PA influenza tetramers. The anti-α1 treatment reduced the proportion of CD8 T cells that were flu NP or PA specific by 40-50% in the BAL, and by 90% in the liver (FIG. 4C). The difference between the BAL and liver results may reflect the ability of the antibody to reach the VLA-1⁺ cells, or that CD8 T cells in the airways have a more limited ability to return to the circulation. In naïve (uninfected) alpha-1 deficient mice, analysis of CD44^{high}/CD62^{low}/CD8+ T cells in the spleen, salivary gland, and liver also revealed 25, reductions in the salivary gland (10% vs. 39%) and liver (8% vs. 26%), and a slight increase of these cells in the spleen (4% vs. 3%/0). Based on these observations, it is understood that in the periphery there are two populations of recirculating memory T cells, one defined by expression of VLA-1 that is resident in the tissue, and another more rapidly recirculating pool that favors secondary lymphoid organs.

Compromised Secondary Immunity via Inhibition of VLA-1:

The number of memory CD8 T cells present in the lung determines the effectiveness of secondary immune protection (Hogan, R. J., et al. (2001) J Immunol 166, 1813-1822). The number is highest immediately following infection, and has been shown to wane with time (Hogan, R. J., et al. (2001) J Immunol 166, 1813-1822). When the number drops below a certain threshold, protection in the lung from secondary infection is reduced (Hogan, R. J., et al. (2001) J Immunol 166, 1813-1822; Hogan, R. J., et al. (2001). J Exp Med 193, 981-986; Liang, S., et al. (1994) J Immunol 152, 1653-1661; Wiley, J. A., et al. (2001) J Immunol 167, 3293-3299). It was hypothesized that if the number of flu-specific memory CD8 T cells in the lung is reduced by blocking VLA-1, then immune control of a secondary infection should be compromised.

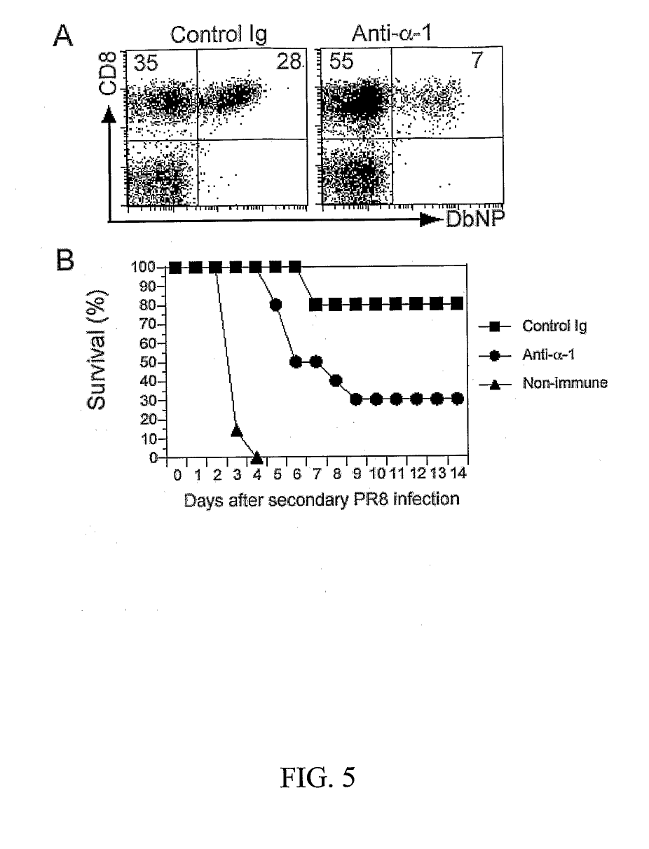

The H₃N₂ HKx31 and H1N1 PR8 strains of influenza A are serologically distinct (Bennink, J., et al. (1978) Immunology 35, 503-509). However, both viruses share the internal nucleoprotein, NP, seen by immune CD8 CTL. Furthermore, these cross-reactive CTL can protect immune mice from lethal challenge with PR8 (Bennink, J., et al. (1978) Immunology 35, 503-509). Three months after primary infection, influenza A/HK/x31 immune mice were pre-treated with al blocking mAb as above (250 μg on alternating days, −5, −3, −1), prior to challenge with a normally lethal dose (3×10⁵ EID₅₀) of influenza A/PR8. At 6 days after PR8 inoculation, the anti-α1 mAb-treated mice exhibited a reduced proportion of the D^{b}NP tetramer⁺ CD8 T cells in the lung (FIG. 5A). More importantly, 70% of the anti-α1 mAb treated mice succumbed to the PR8 infection by day 9, compared to only 20% of the control hamster Ig treated mice, and all of the non-immune animals (FIG. 5B).

Similar VLA-1 blocking studies were performed at 1-2 months and also at more than 6 months after primary X31 infection. At 1-2 months there was no difference in survival between the anti-α1 and control mAb groups, likely reflecting the high number of circulating flu-specific T cells at this early period. Conversely, after 6 months, both the control group and the anti-α1 treated mice had become highly susceptible, presumably because the number of virus-specific CD8 T cells in the lung needed for protection had naturally waned by this time (Hogan, R. J., et al. (2001) J Immunol 166, 1813-1822). These experiments demonstrated that the resident flu-specific CD8 T cells in the lung that provide heterosubtype specific protection were dependent on VLA-1.

Reduction of Tissue Resident Memory CD8 T Cells and Enhanced Susceptibility to Secondary Influenza Infection in VLA-1 Deficient Mice:

Primary infection of alpha-1 deficient mice with influenza A/X31 gave no evidence that these animals were compromised in the ability to control, at least, this primary infection. However, the blocking antibody experiments indicated that inhibition of VLA-1 compromised secondary heterosubtypic influenza immunity. As discussed above, this system is unique in that secondary immune protection is entirely dependent on the number of NP-specific CD8 T cells in the lung at the time of challenge (Hogan, R. J., et al. (2001) J Immunol 166, 1813-1822; Hogan, R. J., et al. (2001). J Exp Med 193, 981-986; Liang, S., et al. (1994) J Immunol 152, 1653-1661; Wiley, J. A., et al. (2001) J Immunol 167, 3293-3299). The absence of VLA-1 on the T cells should decrease the number of flu specific memory T cells in the lung and enhance susceptibility to PR8 challenge

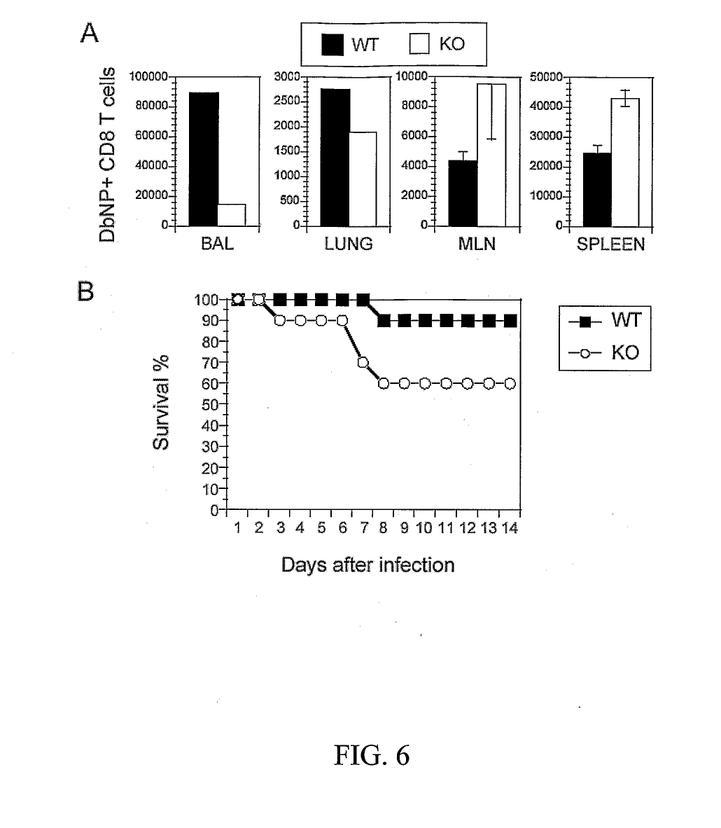

Alpha-1 integrin deficient and wild-type B6 mice were primed with X31 as usual. After recovery, the animals were allowed to rest for 3 months. They were then analyzed for the presence of influenza NP specific CD8 memory populations in the MLN, spleen, BAL, and lung tissue by D^{b}NP tetramer staining. In line with the results obtained with blocking antibodies, mice deficient in alpha-1 integrin had significant decreases in the number of flu specific CD8 T cells, particularly in the BAL, but also in the lung tissue remaining after lavage (FIG. 6A). Conversely, there were significant increases in flu specific memory CD8 T cells in the MLN and spleen indicating no general defect in the ability to generate CD8 memory T cells when alpha-1 integrin is deficient. However, the data does indicate that, in the absence of VLA-1, the T cells are unable to be retained in the long term in the lung.

Based on the reduction in the number of flu specific CD8 T cells in the lung, it follows that the animals should display increased susceptibility to secondary PR8 challenge. In two separate experiments, wild-type and alpha-1 integrin deficient B6 mice were primed with H₃N₂ influenza A/X31 as described above. The animals were rested for 3 months, then inoculated with H1N1 influenza A/PR8 at 3×10⁴ or 3×10⁵ EID₅₀ per mouse. In each experiment, 40% of the alpha-1 deficient mice succumbed to the infection within 8 days of inoculation. One wild-type animal succumbed at the 3×10⁵ EID₅₀ dose of virus. The cumulative data from these experiments are presented in FIG. 6b. Thus, as was the case with the blocking antibodies, the reduction in the number of flu-specific CD8 T cells in the lung that occurs when VLA-1 is inhibited, resulted in enhanced susceptibility to secondary challenge. This enhanced susceptibility occurred in spite of an increased number of flu-specific CD8 T cells in the secondary lymphoid organs. Together, these observations demonstrate that, in the lung, CD8 mediated immune protection from heterosubtypic influenza is dependent on localized virus-specific memory T cells that require VLA-1 to be retained in this extralymphoid compartment. The data further indicate that in this system, which depends on virus-specific CD8 T cells, the memory CD8 T cells in the spleen and MLN are not adequate to provide optimal protection.

An essential element of cellular immune memory and protection is the capacity to establish and retain pathogen specific cells. Following clonal expansion during a primary immune response, populations of memory CD8 T cells become established both in the secondary lymphoid organs, and in a variety of peripheral non-lymphoid tissues. The importance of memory T cells in extralymphoid sites was not known, but it was speculated that they could be important for secondary immunity (Masopust, D., et al. (2001) Science 291, 2413-2417). However, a mechanism to explain how these cells become initially established and are then retained was missing.

The interstitial environment and basement membranes through which lymphocytes must migrate is rich in extracellular matrix (ECM). The most abundant types are the collagens, which in the lung account for 15% of its dry weight (Blankenship, J. W., et al. (1993). Connective Tissue Res. 29, 311-318; van Kuppevelt, T. H., et al. (1995) Int J Biochem & Cell Bio. 27, 775-782). T cells encounter ECM in the secondary lymphoid organs in a way that is fundamentally different from the way they encounter ECM in peripheral tissues. Peripheral tissues are bounded by epithelium on a basement membrane composed of mostly Type IV collagen and laminin (Dustin, M. L., and de Fougerolles, A. R. (2001) Curr Opin Immunol 13, 286-290; Miner, J. H., and Sanes, J. R. (1994) J Cell Bio. 127, 879-891), while the intercellular space in the tissues is largely composed of mostly Type I collagen fibrils (Dustin, M. L., and de Fougerolles, A. R. (2001) Curr Opin Immunol 13, 286-290). Thus T cells entering and migrating through peripheral tissues are constantly in contact with collagen. Histological examination of a variety of extralymphoid tissues shows that most lymphocytes localize near epithelial or endothelial boundaries, at or near basement membrane, rich in Type IV collagen ligand for VLA-1.

The organization of ECM and the accessibility to lymphocytes in lymph nodes on the other hand is vastly different. Collagen fibrils in the lymph nodes are sheathed by fibroblastic reticular cells of the reticular fiber network (Gretz, J. E., et al. (2000) J Exp Med 192, 1425-1440). These reticular conduits for soluble factors and antigens (Gretz, J. E., et al. (1996) J Immunol 157, 495-499; Gretz, J. E., et al. (2000) J Exp Med 192, 1425-1440) are located in the interfollicular areas, distal to the germinal centers (Young, A. J. (1999) Semin Immunol 11, 73-83). Because the collagen in the node is largely hidden from the T cells, they are much less likely to engage ECM.

It has been hypothesized (Dustin, M. L., and de Fougerolles, A. R. (2001) Curr Opin Immunol 13, 286-290) that these differences in the accessibility of collagen between lymphoid and non-lymphoid tissues are related to the way T cells are activated in the two sites. T cells in solution can form clusters with dendritic cells and thereby increase the duration of MHC TCR synapse formation to encourage T cell activation (Dustin, M. L., and de Fougerolles, A. R. (2001) Curr Opin Immunol 13, 286-290). This is essentially the situation in the lymph nodes, and favors the activation of naïve T cells. In the presence of collagen, T cells are stimulated to migrate (Gunzer, M., et al. (2000) Immunity 13, 323-332; Shields, J. M., et al. (1984) Immunology 51, 259-268). In collagen gels and presumably in tissues, T cells form only transient interactions with DC and other cells (Gunzer, M., et al. (2000) Immunity 13, 323-332). This would favor the effector or memory T cells that can be rapidly activated, while discouraging the activation of naïve or resting T cells that require prolonged interaction with MHC and costimulation to become activated (Dustin, M. L., and de Fougerolles, A. R. (2001) Curr Opin Immunol 13, 286-290). Thus the organization of ECM in lymphoid versus non-lymphoid tissues and the expression of collagen-binding integrins on activated versus naïve, or perhaps peripheral versus central memory (Sad, S., and Mosmann, T. R. (1994) J Immunol. 153, 3514-3522; Sallusto, F., et al. (1999) Nature 401, 708-712; Wang, X., and Mosmann, T. (2001) J Exp Med 194, 1069-1080) T cells can be understood in terms of their activation requirements and roles in immunity.

The role of VLA-1 integrin has been investigated in several in vivo models of inflammation using an anti-VLA-1 (anti-alpha-1 integrin) Ha 31/8 mAb that blocks the adhesion of activated T cells to collagen Type I and IV (de Fougerolles, A. R., et al. (2000) J Chin Invest 105, 721-729; Ianaro, A., et al. (2000) Lab Invest 80, 73-80; Mendrick, D. L., et al. (1995) Lab Invest 72, 367-375). Treatment of animals with this antibody inhibits the effector phase inflammatory responses in models of delayed-type hypersensitivity (DTH), contact hypersensitivity (CHS), and arthritis (de Fougerolles, A. R., et al. (2000) J Clin Invest 105, 721-729). No evidence for functional deletion of effector cells was apparent, since treated mice responded normally when the blocking antibody treatment was suspended (de Fougerolles, A. R., et al. (2000) J Clin Invest 105, 721-729). In a rat arthritis model, anti-al mAb was found to block secondary lesion formation, and T cells in the draining lymph nodes had reduced expression of IL2 receptor and proliferative responses (Janaro, A., et al. (2000) Lab Invest 80, 73-80). Similarly, α1-deficient mice were resistant to the induction of arthritis and hypersensitivity reactions (de Fougerolles, A. R., et al. (2000) J Clin Invest 105, 721-729), supporting that the mode of action for the mAb is related to the inhibition of VLA-1 function. Several different cells types can participate in these inflammatory responses, including both CD4 and CD8 T cells. Neither the cell types affected in these studies, nor the effect of the anti-α1 mAb a particular cell type was documented.

In another model of viral infection, LCMV induced delayed type hypersensitivity (DTH), the majority of LCMV-specific CD8 T cells were found to express α1 integrin during the acute (57%) and memory (>80%) phases of the immune response. Similar to the observations here with influenza specific CD8 T cells, the LCMV specific memory CD8 T cells in the spleen were found to have a bimodal distribution of VLA-1 on their surface, again suggesting that two independent populations of memory CD8 T cells can be distinguished by VLA-1. In contrast to the findings described herein, inhibition of α1 by antibody or deletion delayed the primary DTH response in the footpad, though the maximal response was similar to the controls. This response was restored by transfer of the LCMV primed spleen cells directly into the footpad, consistent with the ideas that a certain number of antigen-specific cells are needed at the inflammatory site for an optimal response, and that VLA-1 may be important for regulating this in the periphery.

During influenza infection, as with many virus infections, there is a massive clonal expansion in the number of virus specific CD8 T cells. The number of CTL generated is far in excess of what is necessary to fight off the infection (Tripp, R. A., et al. (1995). J Immunol 155, 2955-2959), and most of the cellular progeny die as the infection is resolved. Most of the death is believed to be by passive apoptosis subsequent to reduced antigen load and IL2 secretion (Van Parijs, L., and Abbas, A. K. (1998) Science 280, 243-248), since mice deficient in either Fas or FasL seem to eliminate excess T cells normally at the end of a response (Lohman, B. L., et al. (1996) J Virol 70, 8199-8203; Razvi, E. S., et al. (1995) Am J Pathol 147, 79-91). In spite of a high rate of apoptosis, some virus-specific CD8 T cells survive and continue to be retained in the lung and other peripheral sites for extended periods of time. The question is what distinguishes those cell destined to survive from those that will die. To survive, they must have a potent mechanism to inhibit the apoptotic pathways inside the cell.

Anchorage-dependent cells such as epithelial and endothelial cells are exquisitely dependent on integrin-mediated attachment to collagen to prevent apoptosis. The death that accompanies detachment of epithelial cells from collagen substrate is termed “anoikis” (Frisch, S. M., and Francis, H. (1994) J Cell Biol 124, 619-626) and has been implicated in preventing damaged or mutated cells from migrating to other tissues. While T cells are clearly not all anchorage dependent, this does not mean that matrix attachment does not promote resistance to apoptosis. Though such a mechanism would be predicted to be distinct at a molecular level from that which drives anoikis.

It was observed that in the lung VLA-1-expressing CD8 T cells are resistant to the apoptosis that follows recovery from influenza infection. The molecular mechanism that conveys this resistance is not currently known, and is under investigation. In a related system, DX5+ influenza-specific CD8 T cells were observed to have an increased susceptibility to apoptosis (Kambayashi, T., et al. (2001) Eur J Immunol 31, 1523-1530). Though the identity of the DX5 ligand was unknown at the time, the DX5 mAb has more recently been shown to recognize the α2 integrin chain (Arase, H., et al. (2001) J Immunol 167, 1141-1144). The large proportion of the flu-specific CD8 T cells also express α2 integrin during the infection, but most of these cells are either lost or down regulate α2 after the virus is cleared. A differential susceptibility to apoptosis related to the expression of either α1 or α2 integrin would indicate an important role for α1 in the establishment of CD8 memory T cells. The predominance of VLA-1+CD8 T cells in the lung and their close proximity to endothelium and epithelium, versus parenchymal locations, indicates that memory CD8 T cells are preferentially and selectively directed to these locations by the pattern of adhesion molecules they express.

Contrary to the observation that α2+ flu-specific T cells are at increased risk of apoptosis, Aoudjit et al (Aoudjit, F., and Vuori, K. (2000) Blood 95, 2044-2051) described that α2β1 mediated attachment to Type I collagen increases resistance of Jurkat T cells to Fas mediated death (Aoudjit, F., and Vuori, K. (2000) Blood 95, 2044-2051). This appeared to be mediated via α2β1 triggered down-modulation of FasL expression. Though this contrasts the situation reported during influenza infection for DX5+ (ie. α2β1+) CD8 T cells (Kambayashi, T., et al. (2001) Eur J Immunol 31, 1523-1530), it may be explained by the different forms of cell death that are occurring. Fas and FasL deficient mice appear to delete excess T cells normally during resolution of viral infections (Lohman, B. L., et al. (1996) J Virol 70, 8199-8203; Razvi, E. S., et al. (1995) Am J Pathol 147, 79-91), suggesting that the deletion is by passive means. α2 would not be protective in this situation, though perhaps α1 offers some protection.

Based on the current observations, it is understood that integrin-mediated ligation of Type I or Type IV collagen conveys resistance to apoptosis of CD8 T cells during acute and memory phases of an immune response, and that in vivo this resistance is conveyed through VLA-1. The resistance of the VLA-1+ subpopulation of virus-primed T cells in the lung promoted the establishment and maintenance of a resident memory population for protection against future secondary encounters with related pathogens.

The second essential element of establishing and maintaining extralymphoid memory T cells would be the capacity to physically retain the cells in the tissue for some period of time. It is well accepted that activated and memory T cells have the capacity to enter inflamed tissues from the circulation (Mackay, C. R., et al. (1992) Eur J Immunol 22, 887-895). This recruitment is largely antigen-specific, though a minority of non-specific memory T cells may be recruited in a bystander fashion, at least early in a response (Topham, D. J., et al. (2001) J Immunol 167, 6983-6990). Additionally, it is understood that acutely activated T cells can enter many, if not all, tissues during an acute immune response, and that some of these cells are retained as memory cells after entry. This finding is supported by the observation that T cells are intimately associated with collagen in the lungs of flu-immune mice, and flu-specific cell enter tissues other than the lung.

The data demonstrate that inhibition of VLA-1 did not inhibit recruitment of influenza-specific CD8 T cells to the lung during infection, indicating that recruitment is not dependent on VLA-1 expression. Once the infection was resolved, a population of predominantly VLA-1+ virus-specific CD8 T cells was recoverable from the lung and other tissues. This phenotype was not unique to the flu-specific cells, since analysis of CD8 T cells in naïve mice revealed a similar, but smaller, population of predominantly VLA-1+ cells that resembled the flu specific cells. This indicated that other immune challenges or pathogens evoke populations of VLA-1+ tissue memory cells. Perhaps most importantly, inhibition of VLA-1 by blocking antibody or genetic deletion compromised this extralymphoid population of memory CD8 T cells in the lung. Though the studies indicate that VLA-1 expression is a stable feature of flu-specific memory CD8 T cells in the lung, the number of recoverable cells has been shown to naturally wane with time (Hogan, R. J., et al. (2001) J Immunol 166, 1813-1822), demonstrating that these memory cells do not persist indefinitely. This indicated that these memory T cells were not static, but either exchanged in a limited fashion with lymphocytes in circulation, or eventually died. In either case, adhesion to collagen increased the half-life of these cells in the tissue, thereby optimizing local secondary immunity for some time after the initial challenge. Acceleration of this loss of tissue memory by inhibition of VLA-1 increased susceptibility to secondary challenge with a virus that the unmanipulated animals can easily resist.

In conclusion, these studies have revealed a new and important role for integrin-mediated matrix attachment in T cell immunity. A mechanism has been described to explain how pathogen-specific CD8 T cells can be established and retained in non-lymphoid tissues. This mechanism has important implications for the design of vaccine strategies to enhance immune protection. It is also an important target for immune therapies aimed at reducing T cell mediated inflammation, such as might occur during allergic reaction. The disruption of immune protection that resulted when the number of virus specific CD8 T cells in the lung was reduced demonstrated that one of the first lines of defense in secondary immunity is the memory T cells in the peripheral tissues. It is understood that the gradual loss of peripheral immunity was linked to the loss of VLA-1 function or expression on tissue-resident memory T cells.

Experimental Procedures:

Animals:

Female C57BL/6 (B6) mice were purchased from Taconic Farms (Germantown, N.Y.) at 6 weeks of age. Congenic Thy1.1+ B6.PL mice were purchased from Jackson Laboratories (Bar Harbor, Me.). Alpha-1 integrin deficient (VLA-1 deficient, VLA-1 KO) (Gardner, H., et al. (1996) Dev. Bio. (Orlando) 175, 301-313) mice backcrossed on to the B6 background were provided by Biogen, Inc. and a colony maintained at the University of Rochester. All animals were housed in the University of Rochester Vivarium facilities under SPF conditions using microisolator technology. Primary inoculation with influenza virus was performed in animals 8-12 weeks of age.

Viruses:

The H3N2 A/Hong Kong/X31 (X31), and H1N1 A/Puerto Rico/8 (PR8) influenza viruses were grown and titered in embryonated chicken eggs and harvested as allantoic fluid preparations (Allan, W., et al. (1990) J Immunol 144, 3980-3986).

Primary and Secondary Influenza Infection:

For primary infections, mice were sedated with avertin (2,2,2-tribromoethanol) prior to intranasal (i.n.) challenge with 10⁵ EID₅₀ of X31 in 30 μl of PBS. For secondary heterosubtypic challenge, X31 immune mice were sedated with avertin and intranasally inoculated with 10³ EID₅₀ of PR8.

Integrin-Specific Antibodies:

The function blocking hamster monoclonal antibody Ha31/8 against α1 (Mendrick, D. L., et al. (1995) Lab Invest 72, 367-375), and the anti-KLH Ha 4/8 control mAb were all affinity purified in an azide free, low endotoxin format at Biogen, Inc. For the in vivo blocking studies, each animal received 250 μg of antibody every other day beginning three days prior to inoculation with virus, and continuing on alternate days throughout the course of the experiment. For flow cytometry, the antibodies were conjugated to Alexa488 or biotin according to the directions of the manufacturer (Molecular Probes, Eugene Oreg.).

Cell Isolations:

Following cardiac perfusion of the mice with PBS, the trachea was canulated and bronchoalveolar lavage (BAL) cells were collected by lavage with 1 ml HBSS three times. These were then resuspended in cMEM and plated in a cell culture treated petri dish for 45 minutes at 37° C. to remove adherant cells. Single cell suspensions were prepared from spleen and lymph nodes by disruption in a dounce homogenizer followed by passage through 90 μm nylon mesh. Splenocytes and peripheral blood lymphocytes (PBL) were depleted of erythrocytes using a buffered ammonium chloride solution (Gey's Solution). Lungs, salivary gland, and kidney where dissociated by pressing though wire mesh and lymphocytes isolated using histopaque 1083 (Sigma Diagnostics, Inc. 1083-1). After perfusion with 5 ml PBS through the hepatic portal vein, livers were excised and the gall bladder removed. Livers were dispersed into single cell suspension by passing through wire mesh, then washed in 40 mls. Each was then digested in 10 ml HBSS containing 2 mg Collagenase IV (Sigma C-5138) and 0.2 mg DNAse I (Sigma DN-25) for 35 minutes at 37° C. with constant rocking. The digested slurry was diluted to 40 ml and subjected to three 30G spins for 3 minutes each to remove debris. The supernatant was then pelleted and resuspended in 26% metrizamide (Sigma M-3383) in HBSS. This was overlaid with 2 ml HBSS and centrifuged at 4° C. for 20 minutes, 1500G, with no brake. Interface was retrieved, washed in 10 ml, and pelleted at 600 g for 5 minutes. All cell counts were obtained by trypan blue exclusion.

Flow Cytometric Analysis:

Lymphocyte populations were stained as aliquots of 2×10⁵ cells with various combinations of mAbs to CD8α (53-6.72 or CT8a), Thy1.1 (OX-7), Thy1.2 (30-H12), CD44 (IM7), and anti-CD62L (MEL-14) conjugated to FITC, phycoerythrin (PE), biotin, APC, or PE-Cy5. The conjugated mabs were purchased from Pharmingen (San Diego, Calif.) or Caltag (Burlingame, Calif.) and are referenced in their current catalogs. Tetrameric complexes of H-2D^{b}/influenza PA₂₂₄₋₂₃₃ (D^{b}PA), H-2 D^{b}/influenza NP₃₆₆₋₃₇₄ (D^{b}NP) and were prepared by the Trudeau Institute Molecular Biology Core Facility and used as described previously (Belz, G. T., et al. (2000) J Virol 74, 3486-3493; Flynn, K. J., et al. (1998) Immunity 8, 683-691). All cells were analyzed with CellQuest software (Becton-Dickinson, San Diego, Calif.), using either a Becton-Dickinson FACScan in three-color mode, or a Becton-Dickinson FACSCalibur in four-color mode.

Intracellular Cytokine Analysis:

Spleen, MLN and non-adherent BAL populations were cultured for 6 h in 96-well round-bottom plates at 5×10⁵ to 8×10⁵ cells per well in complete medium containing 10 μg/ml Brefeldin-A (Epicenter Technologies, Madison, Wis.), with or without 10 μM of the influenza NP₃₆₆₋₃₇₂ or PA₂₂₄₋₂₃₃ peptides. After culture, the cells were placed on ice, washed in PBS/Brefeldin-A (10 μg/ml), stained with a cocktail of anti-α1-Alexa 488 and anti-CD8α-tricolor, washed again, fixed with 1% formaldehyde, permeablized in 0.5% saponin (Sigma, St Louis, Mo.) and stained with anti-IFN-γ-PE (Pharmingen) for 30 min on ice. The lymphocytes were then washed and analyzed in three-color mode with CellQuest software.

TUNEL Assay of Apoptosis:

Lymphocytes were harvested from infected animals at the times indicated, stained for surface marker expression, and then washed in PBS. Stained cells were fixed in 4% paraformaldehyde for 15 min at room temperature, washed and then resuspended in 0.1% Triton X100 in 0.1% sodium citrate to permeabilize the cells. After washing in PBS the cells are then stained by the TUNEL method using the TMR Red In situ Cell Death Detection Kit (Roche Molecular Biologicals, Mannheim, Germany). Positive controls consisted of thymocytes cultured overnight in 10⁻⁵M dexamethasone.

Histology and Immunohistochemistry:

Lungs were inflated with 1:1 mixture of PBS and OCT infused through the trachea and fixed in 0.5% ZnCl₂ (tris-calcium acetate buffer, pH 7.3) for 24 hours. Sections (5 μM) were cut from paraffin-embedded blocks and stained with either hematoxylin and eosin (H&E) for routine histologic examination, trichrome to delineate collagen (Gomori, G. (1950) Am J Clin Path 20, 661-664), or immunohistochemistry (IHC). Slides were deparaffinized, treated with 3% H₂O₂ in H₂O for 6 minutes to quench endogenous peroxidase activity and pretreated for 20 minutes with either heat-induced antigen retrieval (High pH TRS, DAKO) for anti-CD3 or protease K treatment (20 μg/ml at room temperature) for anti-CD8 staining. Endogenous biotin was blocked using the Biotin Block (DAKO, per manufacturer's instructions). Anti-CD3 (1:100, rabbit polyclonal, DAKO) staining was performed for 60 minutes at room temperature, followed by anti-rabbit secondary (DAKO, Rabbit Envision) for 30 minutes. Anti-CD8 staining used 1:80 dilution of monoclonal rat anti-CD8b (Caltag, clone CT-CD8b) for 60 minutes at room temperature, followed by 1:200 dilution of biotinylated rabbit anti-rat (Vector, BA-4000) for 30 minutes at room temperature. DAB (Zymed) was used as the chromogen.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

Allan, W., Tabi, Z., Cleary, A., and Doherty, P. C. (1990). Cellular events in the lymph node and lung of mice with influenza. Consequences of depleting CD4+ T cells. J Immunol 144, 3980-3986.
Aoudjit, F., and Vuori, K. (2000). Engagement of the alpha2beta1 integrin inhibits Fas ligand expression and activation-induced cell death in T cells in a focal adhesion kinase-dependent manner. Blood 95, 2044-2051.
Arase, H., Saito, T., Phillips, J. H., and Lanier, L. L. (2001). Cutting edge: the mouse NK cell-associated antigen recognized by DX5 monoclonal antibody is CD49b (alpha 2 integrin, very late antigen-2). J Immunol 167, 1141-1144.
Bachmann, M. F., Kundig, T. M., Hengartner, H., and Zinkernagel, R. M. (1997). Protection against immunopathological consequences of a viral infection by activated but not resting cytotoxic T cells: T cell memory without “memory T cells”? Proc Natl Acad Sci USA 94, 640-645.
Bank, I., Roth, D., Book, M., Guterman, A., Shnirrer, I., Block, R., Ehrenfeld, M., Langevitz, P., Brenner, H., and Pras, M. (1991). Expression and functions of very late antigen 1 in inflammatory joint diseases. Journal of Clinical Immunology 11, 29-38.
Belkin, V. M., Belkin, A. M., and Koteliansky, V. E. (1990). Human smooth muscle VLA-1 integrin: purification, substrate specificity, localization in aorta, and expression during development. Journal of Cell Biology 111, 2159-2170.
Belz, G. T., Altman, J. D., and Doherty, P. C. (1998). Characteristics of virus-specific CD8(+) T cells in the liver during the control and resolution phases of influenza pneumonia. Proc Natl Acad Sci USA 95, 13812-13817.
Belz, G. T., Xie, W., Altman, J. D., and Doherty, P. C. (2000). A previously unrecognized H-2D(b)-restricted peptide prominent in the primary influenza A virus-specific CD8(+) T-cell response is much less apparent following secondary challenge. J Virol 74, 3486-3493.
Bennink, J., Effros, R. B., and Doherty, P. C. (1978). Influenzal pneumonia: early appearance of cross-reactive T cells in lungs of mice primed with heterologous type A viruses. Immunology 35, 503-509.
Blankenship, J. W., McKinney, B. C., Roos, P. J., and Sandberg, L. B. (1993). Quantitation of lung elastin and collagen in protein and essential fatty acid malnourished rats. Connective Tissue Research 29, 311-318.
Butcher, E. C., and Picker, L. J. (1996). Lymphocyte homing and homeostasis. Science 272, 60-66.
Darzynkiewicz, Z., Bruno, S., Del Bino, G., Gorczyca, W., Hotz, M. A., Lassota, P., and Traganos, F. (1992). Features of apoptotic cells measured by flow cytometry. Cytometry 13, 795-808.
de Fougerolles, A. R., Sprague, A. G., Nickerson-Nutter, C. L., Chi-Rosso, G., Rennert, P. D., Gardner, H., Gotwals, P. J., Lobb, R. R., and Koteliansky, V. E. (2000). Regulation of inflammation by collagen-binding integrins alpha1beta1 and alpha2beta1 in models of hypersensitivity and arthritis. J Clin Invest 105, 721-729.
Doherty, P. C., Hamilton-Easton, A. M., Topham, D. J., Riberdy, J., Brooks, J. W., and Cardin, R. D. (1997). Consequences of viral infections for lymphocyte compartmentalization and homeostasis. Semin Immunol 9, 365-373.
Doherty, P. C., Riberdy, J. M., and Belz, G. T. (2000). Quantitative analysis of the CD8+ T-cell response to readily eliminated and persistent viruses. Philos Trans R Soc Lond B Biol Sci 355, 1093-1101.
Doherty, P. C., Topham, D. J., and Tripp, R. A. (1996). Establishment and persistence of virus-specific CD4+ and CD8+ T cell memory. Immunol Rev 150, 23-44.
Dustin, M. L., and de Fougerolles, A. R. (2001). Reprograming T cells: the role of extracellular matrix in coordination of T cell activation and migration. Curr Opin Immunol 13, 286-290.
Effros, R. B., Bennink, J., and Doherty, P. C. (1978). Characteristics of secondary cytotoxic T-cell responses in mice infected with influenza A viruses. Cell Immunol 36, 345-353.
Effros, R. B., Doherty, P. C., Gerhard, W., and Bennink, J. (1977). Generation of both cross-reactive and virus-specific T-cell populations after immunization with serologically distinct influenza A viruses. J Exp Med 145, 557-568.
Ennis, F. A., Verbonitz, M., Reichelderfer, P., and Daniel, S. (1976). Recombination of influenza A virus strains: effect on pathogenicity. Dev Biol Stand 33, 220-225.
Flynn, K. J., Belz, G. T., Altman, J. D., Ahmed, R., Woodland, D. L., and Doherty, P. C. (1998). Virus-specific CD8+ T cells in primary and secondary influenza pneumonia. Immunity 8, 683-691.
Frisch, S. M., and Francis, H. (1994). Disruption of epithelial cell-matrix interactions induces apoptosis. J Cell Biol 124, 619-626.
Gardner, H., Kreidberg, J., Koteliansky, V., and Jaenisch, R. (1996). Deletion of integrin alpha 1 by homologous recombination permits normal murine development but gives rise to a specific deficit in cell adhesion. Developmental Biology (Orlando) 175, 301-313.
Goldman, R., Harvey, J., and Hogg, N. (1992). VLA-2 is the integrin used as a collagen receptor by leukocytes. European Journal of Immunology 22, 1109-1114.
Gomori, G. (1950). A rapid one-step trichrome stain. Am J Clin Path 20, 661-664.
Gretz, J. E., Kaldjian, E. P., Anderson, A. O., and Shaw, S. (1996). Sophisticated strategies for information encounter in the lymph node: the reticular network as a conduit of soluble information and a highway for cell traffic. J Immunol 157, 495-499.
Gretz, J. E., Norbury, C. C., Anderson, A. O., Proudfoot, A. E., and Shaw, S. (2000). Lymph-borne chemokines and other low molecular weight molecules reach high endothelial venules via specialized conduits while a functional barrier limits access to the lymphocyte microenvironments in lymph node cortex. J Exp Med 192, 1425-1440.
Gunzer, M., Schafer, A., Borgmann, S., Grabbe, S., Zanker, K. S., Brocker, E. B., Kampgen, E., and Friedl, P. (2000). Antigen presentation in extracellular matrix: interactions of T cells with dendritic cells are dynamic, short lived, and sequential. Immunity 13, 323-332.
Hemler, M. E. (1990). VLA proteins in the integrin family: structures, functions, and their role on leukocytes. Annual Review of Immunology 8, 365-400.
Hemler, M. E., Glass, D., Coblyn, J. S., and Jacobson, J. G. (1986). Very late activation antigens on rheumatoid synovial fluid T lymphocytes. Association with stages of T cell activation. Journal of Clinical Investigation 78, 696-702.
Hemler, M. E., and Jacobson, J. G. (1987). Cell matrix adhesion-related proteins VLA-1 and VLA-2: regulation of expression on T cells. J Immunol 138, 2941-2948.
Hemler, M. E., Jacobson, J. G., Brenner, M. B., Mann, D., and Strominger, J. L. (1985). VLA-1: a T cell surface antigen which defines a novel late stage of human T cell activation. Eur J Immunol 15, 502-508.
Hernandez-Pando, R., and Rook, G. A. (1994). The role of TNF-alpha in T-cell-mediated inflammation depends on the Th1/Th2 cytokine balance. Immunology 82, 591-595.
Hogan, R. J., Usherwood, E. J., Zhong, W., Roberts, A. A., Dutton, R. W., Harmsen, A. G., and Woodland, D. L. (2001). Activated antigen-specific CD8+ T cells persist in the lungs following recovery from respiratory virus infections. J Immunol 166, 1813-1822.
Hogan, R. J., Zhong, W., Usherwood, E. J., Cookenham, T., Roberts, A. D., and Woodland, D. L. (2001). Protection from respiratory virus infections can be mediated by antigen-specific CD4(+) T cells that persist in the lungs. J Exp Med 193, 981-986.
Ianaro, A., C₁₋cala, C., Calignano, A., Koteliansky, V., Gotwals, P., Bucci, M., Gerli, R., Santucci, L., Fiorucci, S., and Cirino, G. (2000). Anti-very late antigen-1 monoclonal antibody modulates the development of secondary lesion and T-cell response in experimental arthritis. Lab Invest 80, 73-80.
Kambayashi, T., Assarsson, E., Chambers, B. J., and Ljunggren, H. G. (2001). Expression of the DX5 antigen on CD8+ T cells is associated with activation and subsequent cell death or memory during influenza virus infection. Eur J Immunol 31, 1523-1530.
Kunkel, E. J., and Butcher, E. C. (2002). Chemokines and the tissue-specific migration of lymphocytes. Immunity 16, 1-4.
Laver, W. G., Bischofberger, N., and Webster, R. G. (2000). The origin and control of pandemic influenza. Perspect Biol Med 43, 173-192.
Liang, S., Mozdzanowska, K., Palladino, G., and Gerhard, W. (1994). Heterosubtypic immunity to influenza type A virus in mice. Effector mechanisms and their longevity. J Immunol 152, 1653-1661.
Lohman, B. L., Razvi, E. S., and Welsh, R. M. (1996). T-lymphocyte downregulation after acute viral infection is not dependent on CD95 (Fas) receptor-ligand interactions. J Virol 70, 8199-8203.
Mackay, C. R., Marston, W. L., Dudler, L., Spertini, O., Tedder, T. F., and Hein, W. R. (1992). Tissue-specific migration pathways by phenotypically distinct subpopulations of memory T cells. Eur J Immunol 22, 887-895.
Marshall, D. R., Turner, S. J., Belz, G. T., Wingo, S., Andreansky, S., Sangster, M. Y., Riberdy, J. M., Liu, T., Tan, M., and Doherty, P. C. (2001). Measuring the diaspora for virus-specific CD8+ T cells. Proc Natl Acad Sci USA 98, 6313-6318.
Masopust, D., Vezys, V., Marzo, A. L., and Lefrancois, L. (2001). Preferential localization of effector memory cells in nonlymphoid tissue. Science 291, 2413-2417.
Mendrick, D. L., Kelly, D. M., duMont, S. S., and Sandstrom, D. J. (1995). Glomerular epithelial and mesangial cells differentially modulate the binding specificities of VLA-1 and VLA-2. Lab Invest 72, 367-375.
Miner, J. H., and Sanes, J. R. (1994). Collagen IV alpha 3, alpha 4, and alpha 5 chains in rodent basal laminae: sequence, distribution, association with laminins, and developmental switches. Journal of Cell Biology 127, 879-891.
Munoz, F. M., Galasso, G. J., Gwaltney, J. M., Jr., Hayden, F. G., Murphy, B., Webster, R., Wright, P., and Couch, R. B. (2000). Current research on influenza and other respiratory viruses: II international symposium. Antiviral Res 46, 91-124.
Razvi, E. S., Jiang, Z., Woda, B. A., and Welsh, R. M. (1995). Lymphocyte apoptosis during the silencing of the immune response to acute viral infections in normal, lpr, and Bcl-2-transgenic mice. Am J Pathol 147, 79-91.
Reinhardt, R. L., Khoruts, A., Merica, R., Zell, T., and Jenkins, M. K. (2001). Visualizing the generation of memory CD4 T cells in the whole body. Nature 410, 101-105.
Sad, S., and Mosmann, T. R. (1994). Single IL-2-secreting precursor CD4 T cell can develop into either Th1 or Th2 cytokine secretion phenotype. Journal of mnimunology 153, 3514-3522.
Sado, Y., Kagawa, M., Naito, I., Ueki, Y., Seki, T., Momota, R., Oohashi, T., and Ninomiya, Y. (1998). Organization and expression of basement membrane collagen IV genes and their roles in human disorders. Journal of Biochemistry 123, 767-776.
Sallusto, F., Lenig, D., Forster, R., Lipp, M., and Lanzavecchia, A. (1999). Two subsets of memory T lymphocytes with distinct homing potentials and effector functions [see comments]. Nature 401, 708-712.
Saltini, C., Hemler, M. E., and Crystal, R. G. (1988). T lymphocytes compartmentalized on the epithelial surface of the lower respiratory tract express the very late activation antigen complex VLA-1. Clin Immunol Immunopathol 46, 221-233.
Seki, N., Sudo, Y., Yamane, A., Sugihara, S., Takai, Y., Ishihara, K., Ono, S., Hamaoka, T., Senoh, H., and Fujiwara, H. (1989). Type II collagen-induced murine arthritis. II. Genetic control of arthritis induction is expressed on L3T4+ T cells required for humoral as well as cell-mediated immune responses to type II collagen. Reg Immunol 2, 203-212.
Shields, J. M., Haston, W., and Wilkinson, P. C. (1984). Invasion of collagen gels by mouse lymphoid cells. Immunology 51, 259-268.
Stemme, S., Holm, J., and Hansson, G. K. (1992). T lymphocytes in human atherosclerotic plaques are memory cells expressing CD45RO and the integrin VLA-1. Arteriosclerosis & Thrombosis 12, 206-211.
Takahashi, H., Soderstrom, K., Nilsson, E., Kiessling, R., and Patarroyo, M. (1992). Integrins and other adhesion molecules on lymphocytes from synovial fluid and peripheral blood of rheumatoid arthritis patients. European Journal of Immunology 22, 2879-2885.
Tite, J. P., Hughes-Jenkins, C., O'Callaghan, D., Dougan, G., Russell, S. M., Gao, X. M., and Liew, F. Y. (1990). Anti-viral immunity induced by recombinant nucleoprotein of influenza A virus. II. Protection from influenza infection and mechanism of protection. Immunology 71, 202-207.
Topham, D. J., Castrucci, M. R., Wingo, P. S., Belz, G. T., and Doherty, P. C. (2001). The role of antigen in the localization of naïve, acutely activated, and memory CD8(+) T cells to the lung during influenza pneumonia. J mmunol 167, 6983-6990.
Topham, D. J., and Doherty, P. C. (1998). Clearance of an influenza A virus by CD4+ T cells is inefficient in the absence of B cells. J Virol 72, 882-885.
Tripp, R. A., Hou, S., McMickle, A., Houston, J., and Doherty, P. C. (1995). Recruitment and proliferation of CD8+ T cells in respiratory virus infections. J Immunol 154, 6013-6021.
Tripp, R. A., Sarawar, S. R., and Doherty, P. C. (1995). Characteristics of the influenza virus-specific CD8+ T cell response in mice homozygous for disruption of the H-21Ab gene. J Immunol 155, 2955-2959.
van Kuppevelt, T. H., Veerkamp, J. H., and Timmermans, J. A. (1995). Immunoquantification of type I, III, IV and V collagen in small samples of human lung parenchyma. International Journal of Biochemistry & Cell Biology 27, 775-782.
Van Parijs, L., and Abbas, A. K. (1998). Homeostasis and self-tolerance in the immune system: turning lymphocytes off. Science 280, 243-248.
Wang, X., and Mosmann, T. (2001). In vivo Priming of CD4 T Cells that Produce Interleukin (IL)-2 but not IL-4 or Interferon (IFN)-gamma, and Can Subsequently Differentiate into IL-4- or IFN-gamma-secreting Cells. J Exp Med 194, 1069-1080.
Webster, R. G. (1998). Influenza: an emerging disease. Emerg Infect Dis 4, 436-441.
Wiley, J. A., Hogan, R. J., Woodland, D. L., and Harmsen, A. G. (2001). Antigen-specific CD8(+) T cells persist in the upper respiratory tract following influenza virus infection. J Immunol 167, 3293-3299.
Yewdell, J. W., Bennink, J. R., Smith, G. L., and Moss, B. (1985). Influenza A virus nucleoprotein is a major target antigen for cross-reactive anti-influenza A virus cytotoxic T lymphocytes. Proc Natl Acad Sci USA 82, 1785-1789.
Young, A. J. (1999). The physiology of lymphocyte migration through the single lymph node in vivo. Semin Immunol 11, 73-83.

What is claimed is:

1. A method of assessing the efficacy of an immune response to a selected antigen in a subject comprising
a. introducing into the subject the antigen,
b. collecting a tissue sample from the subject, and
c. detecting the presence of VLA-1+ (positive), antigen-specific T-cells in the sample, the presence of VLA-1+ (positive) antigen-specific T-cells indicating an effective immune response in the subject.

2. The method of claim 1, wherein the antigen is a viral antigen.

3. The method of claim 1, wherein the viral antigen is selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

4. The method of claim 1, wherein the viral antigen is an Influenza-A viral antigen.

5. The method of claim 1, wherein the antigen is a bacterial antigen.

6. The method of claim 5, wherein the bacterial antigen is selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pizeumophila, other Legionella species, Salinonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burtietti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

7. The method of claim 1, wherein the antigen is a fungal antigen.

8. The method of claim 7, wherein the fungal antigen is selected from the group consisting of Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata.

9. The method of claim 1, wherein the antigen is a parasitic antigen.

10. The method of claim 1, wherein the parasitic infection can be selected from the group consisting of Toxoplasma gondii, Plasmodium falciparum, Plasm odium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica.

11. The method of claim 1, wherein the antigen is a cancer-related antigen.

12. The method of claim 11, wherein the antigen is related to a cancer selected from the group consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumors, myelomas, AIDS-related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer.

13. The method of claim 1, wherein the antigen is an Alzheimer's related antigen.

14. The method of claim 1, wherein the antigen is an amyloid antigen.

15. The method of claim 1, wherein the tissue sample is blood.

16. The method of claim 1, wherein the tissue sample is obtained by pulmonary lavage.

17. The method of claim 1, wherein the tissue sample is obtained by tissue biopsy.

18. The method of claim 1, wherein the tissue sample is a non-lymphoid tissue sample.

19. The method of claim 1, wherein the antigen-specific T cells are peripheral memory T cells.

20. The method of claim 1, wherein the tissue sample is collected 6-10 days after the antigen introduction.

21. The method of claim 1, wherein the tissue sample is collected 10-14 days after the antigen introduction.

22. The method of claim 1, wherein the tissue sample is collected 14-21 days after the antigen introduction.

23. The method of claim 1, wherein the tissue sample is collected 21-30 days after the antigen introduction.

24. The method of claim 1, wherein the tissue sample is collected 30-60 days after the antigen introduction.

25. The method of claim 1, wherein the tissue sample is collected 2-6 months after the antigen introduction.

26. The method of claim 1, wherein the subject is a non-primate.

27. The method of claim 1, wherein the subject is a primate.

28. The method of claim 27, wherein the subject is a human.

29. The method of claim 1, wherein the efficacy is measured by immunohistochemistry.

30. The method of claim 1, wherein the efficacy is measured by flow cytometry.

31. The method of claim 1, wherein antigen specificity of the T-cells is detected by positive tetramer staining.

32. The method of claim 1, further comprising detecting the presence of one or more of CD45RO, CD45RA, CD44, CD62L, CD27, and CD43 on the VLA-1+, antigen-specific T-cells.

33. The method of claim 1, further comprising quantifying the level of VLA-1+ antigen-specific T-cells in the sample, an increased level as compared to a control level indicating the sufficiency of the immune response in the subject.

34. A method of screening for an antigen that elicits a sufficient immune response in a subject comprising
a. introducing into the subject the antigen to be tested,
b. collecting a tissue sample from the subject, and
c. measuring VLA-1+ (positive), antigen-specific T-cells in the sample, a high level of VLA-1+ (positive) antigen-specific T-cells as compared to a control sample indicating an antigen that elicits an immune response in the subject.

35. A method a treating a subject with a disease comprising administering to the subject an antigen identified by the method of claim 34, wherein the antigen is related to the disease.

36. A method of isolating from a donor subject VLA-1+ (positive), antigen-specific T-cells comprising
a. introducing into the subject a selected antigen,
b. collecting a tissue sample from the subject, and
c. isolating VLA-1+ (positive), antigen-specific T-cells from the sample.

37. A method a treating a subject with a disease comprising administering to the subject VLA-1+ (positive), antigen-specific T-cells isolated from a donor subject VLA-1+ (positive), antigen-specific T-cells isolated by the method of claim 36.

38. The method of claim 37, wherein the disease is a viral infection.

39. The method of claim 38, wherein the viral infection can be selected from the list of viruses consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian immunodeficiency virus, Human immunodeficiency virus type-1, and Human immunodeficiency virus type-2.

40. The method of claim 38, wherein the virus is Influenza-A virus.

41. The method of claim 37, wherein the disease is a bacterial infection.

42. The method of claim 41, wherein the bacterial infection can be selected from the list of bacterium consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

43. The method of claim 37, wherein the disease is a fungal infection.

44. The method of claim 43, wherein the fungal infection can be selected from the group consisting of Candida albicans, Cryptococcus neoformans, Histoplama capsulatum, Aspergillus fumigatus, Coccidiodes immitis, Paracoccidiodes brasiliensis, Blastomyces dermitidis, Pizeomocystis carnii, Penicillium marneffi, and Alternaria alternata.

45. The method of claim 37, wherein the disease is a parasitic infection.

46. The method of claim 45, wherein the parasitic infection can be selected from the group consisting of Toxoplasma gondii, Plasmodium falciparum, Plasmodium vivax, Plasmodium malariae, other Plasmodium species, Trypanosoma brucei, Trypanosoma cruzi, Leishmania major, other Leishmania species., Schistosoma mansoni, other Schistosoma species, and Entamoeba histolytica.

47. The method of claim 37, wherein the disease is a cancer.

48. The method of claim 47, wherein the cancer is can be selected from the group consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumors, myelomas, AIDS-related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer.

49. The method of claim 37, wherein the disease treated is an inflammatory condition.

50. The methods of claim 49, wherein the inflammatory condition can be selected from the group consisting of asthma, systemic lupus erythematosus, rheumatoid arthritis, reactive arthritis, spondylarthritis, systemic vasculitis, insulin dependent diabetes mellitus, multiple sclerosis, experimental allergic encephalomyelitis, Sjögren's syndrome, graft versus host disease, inflammatory bowel disease including Crohn's disease, ulcerative colitis, ischemia reperfusion injury, myocardial infarction, Alzheimer's disease, transplant rejection (allogeneic and xenogeneic), thermal trauma, any immune complex-induced inflammation, glomerulonephrtis, myasthenia gravis, cerebral lupus, Guillaine-Barre syndrome, vasculitis, systemic sclerosis, anaphylaxis, catheter reactions, atheroma, infertility, thyroiditis, ARDS, post-bypass syndrome, hemodialysis, juvenile rheumatoid, Behcets syndrome, hemolytic anemia, pemphigus, bulbous pemphigoid, stroke, atherosclerosis, and scleroderma.

51. The method of claim 37, wherein the disease treated is Alzheimer's disease

52. The method of claim 37, wherein the antigen-specific T-cells are peripheral memory T cells.
